SEQUENCE LISTING (69 sequences)

SEQ ID NO: 1 - protein, 5 aa, synthetic construct
SNYMS

SEQ ID NO: 2 - protein, 16 aa, synthetic construct
FIYSGGITSYADSVRG

SEQ ID NO: 3 - protein, 11 aa, synthetic construct
RASQGISNWLA

SEQ ID NO: 4 - protein, 5 aa, synthetic construct
GYYWN

SEQ ID NO: 5 - protein, 16 aa, synthetic construct
ESDHSGSTHFNPSLKS

SEQ ID NO: 6 - protein, 17 aa, synthetic construct
RSSQSVLHSSTNKRYLA

SEQ ID NO: 7 - protein, 5 aa, synthetic construct
SYYIH

SEQ ID NO: 8 - protein, 17 aa, synthetic construct
IINPTGGRTIYAQKFQG

SEQ ID NO: 9 - protein, 11 aa, synthetic construct
RASQSVSSYLA

SEQ ID NO: 10 - protein, 5 aa, synthetic construct
SYGMH

SEQ ID NO: 11 - protein, 17 aa, synthetic construct
GITNNGGRTEYADSVKG

SEQ ID NO: 12 - protein, 14 aa, synthetic construct
EDWNGQYYYYAMDV

SEQ ID NO: 13 - protein, 5 aa, synthetic construct
NYAIH

SEQ ID NO: 14 - protein, 17 aa, synthetic construct
VIWSDGDNKYYADSVKG

SEQ ID NO: 15 - protein, 21 aa, synthetic construct
TPWYCTSTNCYTGYYYYGMDV

SEQ ID NO: 16 - protein, 5 aa, synthetic construct
TYAIH

SEQ ID NO: 17 - protein, 17 aa, synthetic construct
WINADNGYTKYSQNFQG

SEQ ID NO: 18 - protein, 21 aa, synthetic construct
TPRYCTSTSCSTGYYYYGMDV

SEQ ID NO: 19 - protein, 5 aa, synthetic construct
TYGMH

SEQ ID NO: 20 - protein, 17 aa, synthetic construct
VISYDGSDKIYADSVKG

SEQ ID NO: 21 - protein, 13 aa, synthetic construct
GPMENYYYYNMDV

SEQ ID NO: 22 - protein, 7 aa, synthetic construct
GFTVSSN

SEQ ID NO: 23 - protein, 5 aa, synthetic construct
YSGGI

SEQ ID NO: 24 - protein, 9 aa, synthetic construct
DDYSNYFDY

SEQ ID NO: 25 - protein, 7 aa, synthetic construct
GGSFSGY

SEQ ID NO: 26 - protein, 5 aa, synthetic construct
DHSGS

SEQ ID NO: 27 - protein, 9 aa, synthetic construct
APDYGDFDH

SEQ ID NO: 28 - protein, 7 aa, synthetic construct
GYTFNSY

SEQ ID NO: 29 - protein, 6 aa, synthetic construct
NPTGGR

SEQ ID NO: 30 - protein, 21 aa, synthetic construct
VPGYCKDTSCQDFYYYYGMDV

SEQ ID NO: 31 - protein, 7 aa, synthetic construct
GLTFSSY

SEQ ID NO: 32 - protein, 6 aa, synthetic construct
TNNGGR

SEQ ID NO: 33 - protein, 14 aa, synthetic construct
EDWNGQYYYYAMDV

SEQ ID NO: 34 - protein, 7 aa, synthetic construct
GFTFTNY

SEQ ID NO: 35 - protein, 6 aa, synthetic construct
WSDGDN

SEQ ID NO: 36 - protein, 21 aa, synthetic construct
TPWYCTSTNCYTGYYYYGMDV

SEQ ID NO: 37 - protein, 7 aa, synthetic construct
GYTFTTY

SEQ ID NO: 38 - protein, 6 aa, synthetic construct
NADNGY

SEQ ID NO: 39 - protein, 21 aa, synthetic construct
TPRYCTSTSCSTGYYYYGMDV

SEQ ID NO: 40 - protein, 7 aa, synthetic construct
GFTFSTY

SEQ ID NO: 41 - protein, 6 aa, synthetic construct
SYDGSD

SEQ ID NO: 42 - protein, 13 aa, synthetic construct
GPMENYYYYNMDV

SEQ ID NO: 43 - protein, 11 aa, synthetic construct
RASQGISNWLA

SEQ ID NO: 44 - protein, 7 aa, synthetic construct
AASSLQS

SEQ ID NO: 45 - protein, 9 aa, synthetic construct
QQAKSFPIT

SEQ ID NO: 46 - protein, 17 aa, synthetic construct
RSSQSVLHSSTNKRYLA

SEQ ID NO: 47 - protein, 7 aa, synthetic construct
WTSTRES

SEQ ID NO: 48 - protein, 9 aa, synthetic construct
QQYYSTPYT

SEQ ID NO: 49 - protein, 11 aa, synthetic construct
RASQSVSSYLA

SEQ ID NO: 50 - protein, 7 aa, synthetic construct
DASNRAT

SEQ ID NO: 51 - protein, 9 aa, synthetic construct
QQRSNWPPT

SEQ ID NO: 52 - protein, 117 aa, synthetic construct
EVQLVESGGGLVQPGGSLRLSCAASGFTVSSNYMSWVRQTPGKGLELVSFIYSGGITSYADSVRGRFTISRDNSKNTLYLQMNSLRAEDTAVYYCARDDYSNYFDYWGQGTLVTVSS

SEQ ID NO: 53 - protein, 107 aa, synthetic construct
DIQMTQSPSSVSASVGDRVTITCRASQGISNWLAWYQQKPGKAPKLLIFAASSLQSGVPSRFSGSGSGTDFTLTISSLQPEDFATYYCQQAKSFPITFGQGTRLEIK

SEQ ID NO: 54 - protein, 117 aa, synthetic construct
EVQLQQWGAGLLKPSETLSLTCAVYGGSFSGYYWNWIRQPPGKGLEWIGESDHSGSTHFNPSLKSRVTISLDTSKNQFSLKLNSVTAADTAVYFCARAPDYGDFDHWGQGILVTVSS

SEQ ID NO: 55 - protein, 113 aa, synthetic construct
DIQMTQSPDSLAVSLGERATINCRSSQSVLHSSTNKRYLAWYQQKPRQPPKLLIYWTSTRESGVPDRFSGSGSGTDFTLTISSLQAEDVAVYFCQQYYSTPYTFGQGTKLEIK

SEQ ID NO: 56 - protein, 130 aa, synthetic construct
QVQLVQSGAEVKKPGASVKVSCKASGYTFNSYYIHWVRQAPGQGLEWLGIINPTGGRTIYAQKFQGRVTMTRDTSTTTVDMELSSLRSEDTAVYYCARVPGYCKDTSCQDFYYYYGMDVWGQGSTVTVSS

SEQ ID NO: 57 - protein, 123 aa, synthetic construct
QVQLVQSGGGLVQPGGSLRLSCSASGLTFSSYGMHWVRQAPGKGLDYVSGITNNGGRTEYADSVKGRFTISRDNSKNTLYLQMSSLRTEDTAVYYCVKEDWNGQYYYYAMDVWGQGTTVTVSS

SEQ ID NO: 58 - protein, 130 aa, synthetic construct
QVQLVQSGGGVVQPGRSLRLSCAASGFTFTNYAIHWVRQAPGKGLEWVAVIWSDGDNKYYADSVKGRFTISRDNSKNTLYLQMNSLRAEDTAVYYCARTPWYCTSTNCYTGYYYYGMDVWGQGTTVTVSS

SEQ ID NO: 59 - protein, 130 aa, synthetic construct
QVQLVQSGAEVKRPGASVKVSCKASGYTFTTYAIHWVRQAPGQRLEWMGWINADNGYTKYSQNFQGRVTITRDTSASTAYMDLSTLRSEDTALYYCARTPRYCTSTSCSTGYYYYGMDVWGQGTTVTVSS

SEQ ID NO: 60 - protein, 122 aa, synthetic construct
QVQLVQSGGGVVQPGRSLRLSCAASGFTFSTYGMHWVRQAPGKGLEWVAVISYDGSDKIYADSVKGRFTISRDNSKNTLFLQMNSLRAEDTAVYYCATGPMENYYYYNMDVWGLGTTVTVSS

SEQ ID NO: 61 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 62 - protein, 122 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGAEYIPAEYFQHWGQGTLVTVSS

SEQ ID NO: 63 - protein, 106 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCQQRPFLPTFGGGTKVEIK

SEQ ID NO: 64 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 65 - protein, 272 aa, Macaca fascicularis
MDPYLLMWGLLTFITVPGCQAELCDDDPPKITHATFKAVAYKEGTMLNCECKRGFRRIKSGSPYMLCTGNSSHSSWDNQCQCTSSAARNTTKQVTPQPEEQKERKTTEMQSQMQLADQVSLPGHCREPPPWENEATERIYHFVVGQTVYYQCVQGYRALHRGPAESVCKMTHGKTRWTQPQLICTGETEPSQFPGEEEPQASPDGLPESETSRLVTTTDFRIQTEVAATMETFIFTTEYQVAVAGCVFLLISVLLLSGLTWQRRQRKNRRTI

SEQ ID NO: 66 - protein, 123 aa, synthetic construct
EVQLVESGGDLVQPRGSLKLSCAASGFTFSSYGMSWVRQTPDKRLELVATINGYGDTTYYPDSVKGRFTISRDNAKNTLYLQMSSLKSEDTAMYFCARDRDYGNSYYYALDYWGQGTSVTVSS

SEQ ID NO: 67 - protein, 106 aa, synthetic construct
QIVLSQSPAILSASPGERVTMTCRASSSVSFMHWLQQKPGSSPKPWIYATSNLASGVSARFSGSGSGTSYSLTITRVEAEDAATYYCQQWSSNPPAFGGGTKLEIK

SEQ ID NO: 68 - protein, 116 aa, synthetic construct
QVQLVQSGAEVKKPGSSVKVSCKASGYTFTSYRMHWVRQAPGQGLEWIGYINPSTGYTEYNQKFKDKATITADESTNTAYMELSSLRSEDTAVYVCARGGGVFDYWGQGTLVTVSS

SEQ ID NO: 69 - protein, 106 aa, synthetic construct
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

(19) United States
(12) Patent Application Publication
(10) Pub. No.: US 2026/0226180 A1
(43) Pub. Date: Aug. 6, 2026

(54) ANTI-IL2RA ANTIBODIES AND USES THEREOF

(71) Applicant: Biocytogen Pharmaceuticals (Beijing) Co., Ltd., Beijing (CN)

(72) Inventors: Yongfei Yang, Beijing (CN); Wenjiao Zhang, Beijing (CN); Shuzhen Cao, Beijing (CN); Zeliang Guan, Beijing (CN); Jing Zhang, Beijing (CN); Yacui Liu, Beijing (CN)

(21) Appl. No.: 19/155,622

(22) PCT Filed: Feb. 21, 2024
(86) PCT No.: PCT/CN2024/077898

§ 371 Date: Aug. 12, 2025

(30) Foreign Application Priority Data
Feb. 22, 2023 (WO) PCT/CN2023/077623

(51) Int. Cl. C07K 16/28; A61P 35/00; C07K 14/705

(52) U.S. Cl. CPC C07K 16/2866; A61P 35/00; C07K 14/7051; C07K 16/2803; C07K 16/2818; C07K 16/2827; C07K 16/2878; C07K 2317/24

(57) ABSTRACT

Provided are anti-IL2RA (interleukin-2 receptor alpha chain) antibodies, antigen-binding fragments, and the uses thereof.

### CLAIM OF PRIORITY

This application claims priority to PCT/CN2023/077623, filed on Feb. 22, 2023. The entire contents of the foregoing application are incorporated herein by reference.

### TECHNICAL FIELD

This disclosure relates to anti-IL2RA (interleukin-2 receptor alpha chain) antibodies and uses thereof.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million.

Recent clinical and commercial success of anticancer antibodies has created great interest in antibody-based therapeutics. There is a need to develop antibodies for use in various antibody-based therapeutics to treat cancers or auto-immune diseases.

### SUMMARY

This disclosure relates to anti-IL2RA antibodies, antigen-binding fragment thereof, and the uses thereof.

In one aspect, the disclosure is related to an antibody or antigen-binding fragment thereof that binds to interleukin-2 receptor alpha chain (IL2RA), comprising: a heavy chain variable region (VH) comprising complementarity determining regions (CDRs) 1, 2, and 3, in some embodiments, the VH CDRI region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 43, 44, 45, respectively;
(2) the selected VH CDRs 1, 2, 3 amino acid sequences are set forth in SEQ ID NOs: 4, 5, 6, respectively, and the selected VL CDRs 1, 2, 3 amino acid sequences are set forth in SEQ ID NOs: 46, 47, 48, respectively;
(3) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 49, 50, 51, respectively;
(4) the selected VH CDRs 1, 2, 3 amino acid sequences are set forth in SEQ ID NOs: 10, 11, 12, respectively, and the selected VL CDRs 1, 2, 3 amino acid sequences are set forth in SEQ ID NOs: 49, 50, 51, respectively;
(5) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 49, 50, 51, respectively;
(6) the selected VH CDRs 1, 2, 3 amino acid sequences are set forth in SEQ ID NOs: 16, 17, 18, respectively, and the selected VL CDRs 1, 2, 3 amino acid sequences are set forth in SEQ ID NOs: 49, 50, 51, respectively;
(7) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 49, 50, 51, respectively;
(8) the selected VH CDRs 1, 2, 3 amino acid sequences are set forth in SEQ ID NOs: 22, 23, 24, respectively, and the selected VL CDRs 1, 2, 3 amino acid sequences are set forth in SEQ ID NOs: 43, 44, 45, respectively;
(9) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 46, 47, 48, respectively;
(10) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 49, 50, 51, respectively;
(11) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 49, 50, 51, respectively;
(12) the selected VII CDRs 1, 2, 3 amino acid sequences are set forth in SEQ ID NOs: 34, 35, 36, respectively, and the selected VL CDRs 1, 2, 3 amino acid sequences are set forth in SEQ ID NOs: 49, 50, 51, respectively;
(13) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 49, 50, 51, respectively; and
(14) the selected VH CDRs 1, 2, 3 amino acid sequences are set forth in SEQ ID NOs: 40, 41, 42, respectively, and the selected VL CDRs 1, 2, 3 amino acid sequences are set forth in SEQ ID NOs: 49, 50, 51, respectively.

In some embodiments, the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 43, 44, and 45, respectively according to Kabat definition. In some embodiments, the VH comprises CDRs 1, 2, 3 with the amino acid sequences set forth in SEQ ID NOs: 4, 5, and 6, respectively, and the VL comprises CDRs 1, 2, 3 with the amino acid sequences set forth in SEQ ID NOs: 46, 47, and 48, respectively according to Kabat definition. In some embodiments, the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 49, 50, and 51, respectively according to Kabat definition. In some embodiments, the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 49, 50, and 51, respectively according to Kabat definition. In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 49, 50, and 51, respectively according to Kabat definition. In some embodiments, the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 49, 50, and 51, respectively according to Kabat definition. In some embodiments, the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 49, 50, and 51, respectively according to Kabat definition.

In some embodiments, the antibody or antigen-binding fragment specifically binds to human IL2RA or monkey IL2RA. In some embodiments, the antibody or antigen-binding fragment is a human or humanized antibody or antigen-binding fragment thereof (e.g., a human IgG1 antibody). In some embodiments, the antibody or antigen-binding fragment is a single-chain variable fragment (scFv) or a multi-specific antibody (e.g., a bispecific antibody).

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and in some embodiments, the VH, when paired with a light chain variable region (VL) comprising the amino acid sequence set forth in SEQ ID NO: 53 binds to IL2RA;
(2) an immunoglobulin light chain or a fragment thereof comprising a VL comprising CDRs 1, 2, and 3 comprising the amino acid sequences set forth in SEQ ID NOs: 43, 44, and 45, respectively, and in some embodiments, the VL, when paired with a VH comprising the amino acid sequence set forth in SEQ ID NO: 52 binds to IL2RA;
(3) an immunoglobulin heavy chain or a fragment thereof comprising a heavy chain variable region (VH) comprising CDRs 1, 2, and 3 comprising the amino acid sequences set forth in SEQ ID NOs: 4, 5, and 6, respectively, and in some embodiments, the VH, when paired with a light chain variable region (VL) comprising the amino acid sequence set forth in SEQ ID NO: 55 binds to IL2RA;
(4) an immunoglobulin light chain or a fragment thereof comprising a VL comprising CDRs 1, 2, and 3 comprising the amino acid sequences set forth in SEQ ID NOs: 46, 47, and 48, respectively, and in some embodiments, the VL, when paired with a VH comprising the amino acid sequence set forth in SEQ ID NO: 54 binds to IL2RA;
(5) an immunoglobulin heavy chain or a fragment thereof comprising a heavy chain variable region (VH) comprising CDRs 1, 2, and 3 comprising the amino acid sequences set forth in SEQ ID NOs: 7, 8, and 9, respectively, and in some embodiments, the VH, when paired with a light chain variable region (VL) comprising the amino acid sequence set forth in SEQ ID NO: 61 binds to IL2RA;
(6) an immunoglobulin light chain or a fragment thereof comprising a VL comprising CDRs 1, 2, and 3 comprising the amino acid sequences set forth in SEQ ID NOs: 49, 50, and 51, respectively, and in some embodiments, the VL, when paired with a VH comprising the amino acid sequence set forth in SEQ ID NO: 56 binds to IL2RA;
(7) an immunoglobulin heavy chain or a fragment thereof comprising a heavy chain variable region (VH) comprising CDRs 1, 2, and 3 comprising the amino acid sequences set forth in SEQ ID NOs: 10, 11, and 12, respectively, and in some embodiments, the VH, when paired with a light chain variable region (VL) comprising the amino acid sequence set forth in SEQ ID NO: 61 binds to IL2RA;
(8) an immunoglobulin light chain or a fragment thereof comprising a VL comprising CDRs 1, 2, and 3 comprising the amino acid sequences set forth in SEQ ID NOs: 49, 50, and 51, respectively, and in some embodiments, the VL, when paired with a VH comprising the amino acid sequence set forth in SEQ ID NO: 57 binds to IL2RA;
(9) an immunoglobulin heavy chain or a fragment thereof comprising a heavy chain variable region (VH) comprising CDRs 1, 2, and 3 comprising the amino acid sequences set forth in SEQ ID NOs: 13, 14, and 15, respectively, and in some embodiments, the VH, when paired with a light chain variable region (VL) comprising the amino acid sequence set forth in SEQ ID NO: 61 binds to IL2RA;
(10) an immunoglobulin light chain or a fragment thereof comprising a VL comprising CDRs 1, 2, and 3 comprising the amino acid sequences set forth in SEQ ID NOs: 49, 50, and 51, respectively, and in some embodiments, the VL, when paired with a VH comprising the amino acid sequence set forth in SEQ ID NO: 58 binds to IL2RA;
(11) an immunoglobulin heavy chain or a fragment thereof comprising a heavy chain variable region (VH) comprising CDRs 1, 2, and 3 comprising the amino acid sequences set forth in SEQ ID NOs: 16, 17, and 18, respectively, and in some embodiments, the VH, when paired with a light chain variable region (VL) comprising the amino acid sequence set forth in SEQ ID NO: 61 binds to IL2RA;
(12) an immunoglobulin light chain or a fragment thereof comprising a VL comprising CDRs 1, 2, and 3 comprising the amino acid sequences set forth in SEQ ID NOs: 49, 50, and 51, respectively, and in some embodiments, the VL, when paired with a VH comprising the amino acid sequence set forth in SEQ ID NO: 59 binds to IL2RA;
(13) an immunoglobulin heavy chain or a fragment thereof comprising a heavy chain variable region (VH) comprising CDRs 1, 2, and 3 comprising the amino acid sequences set forth in SEQ ID NOs: 19, 20, and 21, respectively, and in some embodiments, the VH, when paired with a light chain variable region (VL) comprising the amino acid sequence set forth in SEQ ID NO: 61 binds to IL2RA;
(14) an immunoglobulin light chain or a fragment thereof comprising a VL comprising CDRs 1, 2, and 3 comprising the amino acid sequences set forth in SEQ ID NOs: 49, 50, and 51, respectively, and in some embodiments, the VL, when paired with a VH comprising the amino acid sequence set forth in SEQ ID NO: 60 binds to IL2RA;
(15) an immunoglobulin heavy chain or a fragment thereof comprising a heavy chain variable region (VH) comprising CDRs 1, 2, and 3 comprising the amino acid sequences set forth in SEQ ID NOs: 22, 23, and 24, respectively, and in some embodiments, the VH, when paired with a light chain variable region (VL) comprising the amino acid sequence set forth in SEQ ID NO: 53 binds to IL2RA;
(16) an immunoglobulin heavy chain or a fragment thereof comprising a heavy chain variable region (VH) comprising CDRs 1, 2, and 3 comprising the amino acid sequences set forth in SEQ ID NOs: 25, 26, and 27, respectively, and in some embodiments, the VH, when paired with a light chain variable region (VL) comprising the amino acid sequence set forth in SEQ ID NO: 55 binds to IL2RA;
(17) an immunoglobulin heavy chain or a fragment thereof comprising a heavy chain variable region (VH) comprising CDRs 1, 2, and 3 comprising the amino acid sequences set forth in SEQ ID NOs: 28, 29, and 30, respectively, and in some embodiments, the VH, when paired with a light chain variable region (VL) comprising the amino acid sequence set forth in SEQ ID NO: 61 binds to IL2RA;
(18) an immunoglobulin heavy chain or a fragment thereof comprising a heavy chain variable region (VH) comprising CDRs 1, 2, and 3 comprising the amino acid sequences set forth in SEQ ID NOs: 31, 32, and 33, respectively, and in some embodiments, the VH, when paired with a light chain variable region (VL) comprising the amino acid sequence set forth in SEQ ID NO: 61 binds to IL2RA;
(19) an immunoglobulin heavy chain or a fragment thereof comprising a heavy chain variable region (VH) comprising CDRs 1, 2, and 3 comprising the amino acid sequences set forth in SEQ ID NOs: 34, 35, and 36, respectively, and in some embodiments, the VH, when paired with a light chain variable region (VL) comprising the amino acid sequence set forth in SEQ ID NO: 61 binds to IL2RA;
(20) an immunoglobulin heavy chain or a fragment thereof comprising a heavy chain variable region (VH) comprising CDRs 1, 2, and 3 comprising the amino acid sequences set forth in SEQ ID NOs: 37, 38, and 39, respectively, and in some embodiments, the VH, when paired with a light chain variable region (VL) comprising the amino acid sequence set forth in SEQ ID NO: 61 binds to IL2RA; or
(21) an immunoglobulin heavy chain or a fragment thereof comprising a heavy chain variable region (VH) comprising CDRs 1, 2, and 3 comprising the amino acid sequences set forth in SEQ ID NOs: 40, 41, and 42, respectively, and in some embodiments, the VH, when paired with a light chain variable region (VL) comprising the amino acid sequence set forth in SEQ ID NO: 61 binds to IL2RA.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively, or SEQ ID NOs: 22, 23, and 24, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 43, 44, and 45, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4, 5, and 6, respectively, or SEQ ID NOs: 25, 26, and 27, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 46, 47, and 48, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively, or SEQ ID NOs: 28, 29, and 30, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10, 11, and 12, respectively, or SEQ ID NOs: 31, 32, and 33, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively, or SEQ ID NOs: 34, 35, and 36, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16, 17, and 18, respectively, or SEQ ID NOs: 37, 38, and 39, respectively. In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19, 20, and 21, respectively, or SEQ ID NOs: 40, 41, and 42, respectively. In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 49, 50, and 51, respectively.

In some embodiments, the VH when paired with a VL specifically binds to human IL2RA, or the VL when paired with a VH specifically binds to human IL2RA or monkey IL2RA. In some embodiments, the immunoglobulin heavy chain or the fragment thereof is a human or humanized immunoglobulin heavy chain or a fragment thereof (e.g., a human IgG1 heavy chain or a fragment thereof), and the immunoglobulin light chain or the fragment thereof is a human or humanized immunoglobulin light chain or a fragment thereof. In some embodiments, the nucleic acid encodes a single-chain variable fragment (scFv), a multi-specific antibody (e.g., a bispecific antibody) or a chimeric antigen receptor (CAR). In some embodiments, the nucleic acid is cDNA.

In one aspect, the disclosure is related to a vector comprising one or more of the nucleic acids described herein. In one aspect, the disclosure is related to a vector comprising two of the nucleic acids described herein, in some embodiments, the vector encodes the VH region and the VL region that together bind to IL2RA. In one aspect, the disclosure is related to a pair of vectors, in some embodiments, each vector comprises one of the nucleic acids described herein, in some embodiments, together the pair of vectors encodes the VH region and the VL region that together bind to IL2RA.

In one aspect, the disclosure is related to a cell comprising the vector or the pair of vectors described herein. In some embodiments, the cell is a CHO cell. In one aspect, the disclosure is related to a cell comprising one or more of the nucleic acids described herein. In one aspect, the disclosure is related to a cell comprising two of the nucleic acids described herein. In some embodiments, the two nucleic acids together encode the VH region and the VL region that together bind to IL2RA.

In one aspect, the disclosure is related to a method of producing an antibody or an antigen-binding fragment thereof, the method comprising (a) culturing the cell described herein under conditions sufficient for the cell to produce the antibody or the antigen-binding fragment; and (b) collecting the antibody or the antigen-binding fragment produced by the cell.

In one aspect, the disclosure is related to an antibody or antigen-binding fragment thereof that binds to IL2RA comprising a heavy chain variable region (VH) comprising an amino acid sequence that is at least 80% identical to a selected VII sequence, and a light chain variable region (VL) comprising an amino acid sequence that is at least 80% identical to a selected VL sequence, in some embodiments, the selected VH sequence and the selected VL sequence are one of the following: (1) the selected VH sequence is SEQ ID NO: 52, and the selected VL sequence is SEQ ID NO: 53; (2) the selected VH sequence is SEQ ID NO: 54, and the selected VL sequence is SEQ ID NO: 55; (3) the selected VH sequence is SEQ ID NO: 56, and the selected VL sequence is SEQ ID NO: 61; (4) the selected VH sequence is SEQ ID NO: 57, and the selected VL sequence is SEQ ID NO: 61; (5) the selected VH sequence is SEQ ID NO: 58, and the selected VL sequence is SEQ ID NO: 61; (6) the selected VH sequence is SEQ ID NO: 59, and the selected VL sequence is SEQ ID NO: 61; and (7) the selected VH sequence is SEQ ID NO: 60, and the selected VL sequence is SEQ ID NO: 61.

In some embodiments, the VH comprises the sequence of SEQ ID NO: 52 and the VL comprises the sequence of SEQ ID NO: 53. In some embodiments, the VH comprises the sequence of SEQ ID NO: 54 and the VL comprises the sequence of SEQ ID NO: 55. In some embodiments, the VH comprises the sequence of SEQ ID NO: 56 and the VL comprises the sequence of SEQ ID NO: 61. In some embodiments, the VH comprises the sequence of SEQ ID NO: 57 and the VL comprises the sequence of SEQ ID NO: 61. In some embodiments, the VH comprises the sequence of SEQ ID NO: 58 and the VL comprises the sequence of SEQ ID NO: 61. In some embodiments, the VH comprises the sequence of SEQ ID NO: 59 and the VL comprises the sequence of SEQ ID NO: 61. In some embodiments, the VH comprises the sequence of SEQ ID NO: 60 and the VL comprises the sequence of SEQ ID NO: 61. In some embodiments, the antibody or antigen-binding fragment specifically binds to human IL2RA or monkey IL2RA. In some embodiments, the antibody or antigen-binding fragment is a human or humanized antibody or antigen-binding fragment thereof. In some embodiments, the antibody or antigen-binding fragment is a single-chain variable fragment (scFv) or a multi-specific antibody (e.g., a bispecific antibody).

In one aspect, the disclosure is related to an antibody or antigen-binding fragment thereof that cross-competes with the antibody or antigen-binding fragment thereof described herein.

In one aspect, the disclosure is related to an antibody or antigen-binding fragment thereof that binds to IL2RA comprising a heavy chain variable region (VH) comprising VH CDR1, VH CDR2, and VH CDR3 that are identical to VH CDR1, VII CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following: (1) the selected VH sequence is SEQ ID NO: 52, and the selected VL sequence is SEQ ID NO: 53; (2) the selected VH sequence is SEQ ID NO: 54, and the selected VL sequence is SEQ ID NO: 55; (3) the selected VH sequence is SEQ ID NO: 56, and the selected VL sequence is SEQ ID NO: 61; (4) the selected VH sequence is SEQ ID NO: 57, and the selected VL sequence is SEQ ID NO: 61; (5) the selected VH sequence is SEQ ID NO: 58, and the selected VL sequence is SEQ ID NO: 61; (6) the selected VH sequence is SEQ ID NO: 59, and the selected VL sequence is SEQ ID NO: 61; and (7) the selected VH sequence is SEQ ID NO: 60, and the selected VL sequence is SEQ ID NO: 61.

In one aspect, the disclosure is related to an antibody-drug conjugate comprising the antibody or antigen-binding fiagment thereof described herein covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof or the antibody-drug conjugate described herein, to the subject. In some embodiments, the subject has a solid tumor, brain cancer, lung cancer, melanoma, prostate cancer, esophageal squamous cell carcinoma, leukemia, lymphoma, ovarian cancer, colorectal cancer, head and neck cancer, bladder cancer, renal cancer and breast cancer. In some embodiments, the method described herein further comprises administering a therapeutically effective amount of an anti-OX40 antibody, an anti-PD1 antibody, an anti-PDL1 antibody, an anti-PDL2 antibody, an anti-LAG-3 antibody, an anti-TIGIT antibody, an anti-CTLA-4 antibody, an anti-GITR antibody, an anti-TIM-3 antibody, an anti-4-1BB antibody, and/or an anti-CD40 antibody, to the subject.

In one aspect, the disclosure is related to a method of decreasing the rate of tumor growth, the method comprising contacting a tumor cell with an effective amount of a composition comprising an antibody or antigen-binding fragment thereof or the antibody-drug conjugate described herein.

In one aspect, the disclosure is related to a method of killing a tumor cell, the method comprising contacting a tumor cell with an effective amount of a composition comprising the antibody or antigen-binding fragment thereof or the antibody-drug conjugate described herein.

In one aspect, the disclosure is related to a pharmaceutical composition comprising the antibody or antigen-binding fragment thereof described herein and a pharmaceutically acceptable carrier. In one aspect, the disclosure is related to a pharmaceutical composition comprising the antibody drug conjugate described herein, and a pharmaceutically acceptable carrier.

In one aspect, the disclosure is related to a chimeric antigen receptor (CAR) comprising the antibody or antigen-binding fragment thereof described herein.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as head and neck, respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, glioma and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., IL2RA) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a IL2RA molecule may be referred to as a IL2RA-specific antibody or an anti-IL2RA antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B show the blocking effect of the binding between IL2RA ligand and IL2RA in the presence of an anti-IL2RA antibody 11C3, 11C12, 11D2, 13F9, 14C2 (FIG. 1A); 5D9, 7B5 (FIG. 1B). Daclizumab analog was used as a control.

FIGS. 2A-2B show the blocking effect of the binding between mouse IL2 protein and IL2 reporter cells, in the presence of an anti-IL2RA antibody 11C3, 13F9, 11D2, 14C2, 11C12 (FIG. 2A); 5D9, 7B5 (FIG. 2B) at a concentration of 100 μg/mL, 33.3 μg/mL, or 11.1 μg/mL. Daclizumab analog was used as a control.

FIGS. 3A-3B show the blocking effect of STAT5 phosphorylation in human CD3⁺ T cells that were incubated with an anti-IL2RA antibody 11C12, 11D2, 11C3, 13F9, 14C2 (FIG. 3A); 7B5, 5D9 (FIG. 3B) and human IL2 (hIL2). Daclizumab analog was used as a pSTAT5 blocking antibody control. 7G7B6 analog-SI was used as a pSTAT5 non-blocking antibody control.

FIG. 4 shows the tumor volume over time in B-hIL2RA mice that were injected with MC38 cancer cells and treated with PBS (G1), 3 mg/kg 7B5 (G2), 10 mg/kg 7B5 (G3), 3 mg/kg 5D9 (G4), 10 mg/kg 5D9 (G5), 3 mg/kg 7G7B6 analog (G6), or 10 mg/kg 7G7B6 analog (G7).

FIG. 5 shows the tumor volume over time in B-hIL2RA mice that were injected with MC38 cancer cells and treated with PBS (G1), 10 mg/kg 7B5 (G2), 10 mg/kg 5D9 (G3), 10 mg/kg 7G7B6 analog-SI (G4), or 10 mg/kg 7G7B6 analog (G5).

FIG. 6 shows the tumor volume over time in B-hIL2RA mice that were injected with MC38 cancer cells and treated with PBS (G1), 10 mg/kg 7G7B6 analog-SI (G2), 10 mg/kg 11C3 (G3), 10 mg/kg 13F9 (G4), 10 mg/kg 11D2 (G5), 10 mg/kg 11C12 (G6), or 10 mg/kg 14C2 (G7).

FIG. 7 lists Kabat CDR sequences for anti-IL2RA antibodies.

FIG. 8 lists Chothia CDR sequences for anti-IL2RA antibodies.

FIG. 9 lists sequences of the heavy chain variable regions and light chain variable regions of the anti-IL2RA antibodies.

FIG. 10 lists amino acids sequences discussed in the disclosure.

### DETAILED DESCRIPTION

Interleukin-2 (IL-2) is a cytokine capable of sustaining the proliferative potential of T-lymphocytes. IL-2 was determined to be the primary growth factor in activated T-lymphocytes, which is capable of driving clonal expansion and effector cell maturation. IL-2 stimulation can also lead to the growth of natural killer (NK) cells. IL-2 mediates its biologic effects via the IL-2 receptor (IL-2R) complex. IL-2R is comprised of three distinct subunits: alpha, beta, and gamma chains. IL-2R is related to proliferation of activated CD4⁻, CD8⁻, CD4⁺8⁺, CD4⁺, and CD8⁺ T cells. The importance of the IL2RA is demonstrated by its high affinity for IL-2. Signaling pathways involving IL-2R include Jak3-dependent activation of Jak1, which has been shown to be the key proliferative signal in fibroblasts. The elevated expression of sIL2RA and IL2RA protein in tumor cells has been detected in a vast array of cancers, e.g., lung cancer, melanoma, prostate cancer, esophageal squamous cell carcinoma, leukemia, lymphoma, ovarian cancer, colorectal cancer, and breast cancer. Additionally, elevated mRNA and protein expression of IL2RA has been identified in many solid tumor types, such as ovarian, lung, head-and-neck, and breast. Thus, anti-IL2RA antibodies can be potentially used as cancer therapies.

The present disclosure provides examples of antibodies, antigen-binding fragment thereof, that bind to IL2RA (interleukin-2 receptor alpha chain).

### IL2RA

IL2RA (interleukin-2 receptor alpha chain, or CD25) is a type I transmembrane protein present on activated T cells, activated B cells, some thymocytes, myeloid precursors, oligodendrocytes, etc. Though IL2RA has been used as a marker to identify CD4+FoxP3+ regulatory T cells in mice, it has been found that a large proportion of resting memory T cells constitutively express IL2RA in humans. IL2RA is expressed in most B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Its soluble form, called sIL-2R may be elevated in these diseases and is occasionally used to track disease progression.

The interleukin-2 (IL-2) receptor is formed by the α (IL-2RA, CD25), β (IL-2RB, CD122) and γ common (IL-2RG, CD132) subunits, and plays a vital role in maintaining the immune system. The interleukin 2 (IL2) receptor alpha (IL2RA) and beta (IL2RB) chains, together with the common gamma chain (IL2RG), constitute the high-affinity IL2 receptor. Homodimeric alpha chains (IL2RA) result in low-affinity receptor, while homodimeric beta (IL2RB) chains produce a medium-affinity receptor. The high-affinity receptor for IL-2 incorporates all three chains (α, β, and γ) and is present on activated T cells, activated B cells, and Treg cells. The medium-affinity receptor consists of the gamma and beta chains only, and is expressed on NK cells, as well as resting T and B cells. The low-affinity receptor consists of the alpha chain and is expressed on dendritic cells.

Among the IL-2 receptors, IL2RA is a unique subunit that exclusively binds IL-2, while CD132 binds the common ye family cytokines (IL-4, IL-7, IL-9, IL-15 and IL-21), and the CD122 subunit binds IL-15. IL2RA is constitutively expressed at high levels by regulatory T cells (Tregs), and enables them to be the first responders to IL-2 during an immune response and promotes the transcription of FOXP3 by amplifying IL-2 signaling in a STAT5-dependent fashion. Interestingly, single nucleotide polymorphism (SNP) studies of the IL2RA gene have been associated with several forms of autoimmunity demonstrating that IL-2 signaling via IL2RA is an important axis in regulating tolerance. IL2RA is also critical for effector T cell expansion in response to IL-2 immediately after antigenic stimulation.

The formation of the high-affinity quaternary IL-2/IL-2R complex leads to signal transduction through the tyrosine kinases Jak1 and Jak3, which are associated with IL-2Rβ and γc, respectively. Three tyrosine residues within the cytoplasmic tail of IL-2Rβ are phosphorylated to promote recruitment of the adaptor She (Y338 human; Y341 mouse), leading to activation of the MAPK and PI-3K kinase pathways, and predominately the Stat5 transcription factor (Y392 and Y510 human; Y398 and Y505 mouse), resulting in Stat5-dependent gene regulation. The quaternary IL-2-IL-2R complex is rapidly internalized, where IL-2, IL-2Rβ, and γc are rapidly degraded, but IL-2Rα is recycled to the cell surface. Thus, those functional activities that require sustained TL-2R signaling require a continued source of IL-2 to engage IL-2Rα and form additional IL-2-IL-2R signaling complexes.

IL2RA is a protein that in humans is encoded by the IL2RA gene. Normally an integral-membrane protein, soluble IL2RA has been isolated and determined to result from extracellular proteolysis. Alternately-spliced IL2RA mRNAs have been isolated, but the significance of each is currently unknown. Mutations in this gene are associated with interleukin 2 receptor alpha deficiency.

A detailed description of IL2RA and its function can be found, e.g., in Goudy, Kevin, et al., “Human IL2RA null mutation mediates immunodeficiency with lymphoproliferation and autoimmunity.” Clinical Immunology 146.3 (2013): 248-261; Kuhn, Deborah J., and Q. Ping Dou. “The role of interleukin-2 receptor alpha in cancer.” Front Biosci 10 (2005): 1462-1474; Malek, Thomas R., and Iris Castro. “Interleukin-2 receptor signaling: at the interface between tolerance and immunity.” Immunity 33.2 (2010): 153-165; and Jiang et al., “Role of IL-2 in cancer immunotherapy.” Oncoimmunology 5.6 (2016): e1163462; each of which is incorporated by reference in its entirety.

The present disclosure provides anti-IL2RA antibodies, antigen-binding fragments thereof, and methods of using these anti-IL2RA antibodies and antigen-binding fragments to inhibit tumor growth and to treat various diseases, including e.g., cancer.

### Anti-IL2RA Antibodies and Antigen-Binding Fragments

The disclosure provides antibodies and antigen-binding fragments thereof that specifically bind to IL2RA (e.g., human IL2RA). The antibodies and antigen-binding fragments described herein are capable of binding to IL2RA. In some embodiments, these antibodies do not block the binding of human IL2RA to IL2RA ligand (e.g., human IL2). In some embodiments, these antibodies do not block the binding of IL2 protein and IL2 reporter cells. In some embodiments, these antibodies do not significantly inhibit human IL2-induced STAT5 phosphorylation. In some embodiments, these antibodies do not block the IL2/IL2RA signaling pathway thus increase immune response. In some embodiments, these antibodies can initiate complement-dependent cytotoxicity (CDC) or antibody-dependent cellular cytotoxicity (ADCC). In some embodiments, these antibodies bind to cells expressing IL2RA.

The disclosure provides, e.g., anti-IL2RA antibodies 5D9, 7B5, 11C3, 1IC12, 11D2, 13F9 and 14C2, the modified antibodies thereof, including, e.g., chimeric antibodies, humanized antibodies, and human antibodies.

The CDR sequences for 5D9, and 5D9 derived antibodies (e.g., humanized antibodies) include CDRs of the heavy chain variable domain, SEQ ID NOs: 1, 2, 3, and CDRs of the light chain variable domain, SEQ ID NOs: 43, 44, 45, as defined by Kabat definition. The CDRs can also be defined by Chothia system. Under the Chothia definition, the CDR sequences of the heavy chain variable domain are set forth in SEQ ID NOs: 22, 23, 24, and CDR sequences of the light chain variable domain are set forth in SEQ ID NOs: 43, 44, 45.

The CDR sequences for 7B5, and 7B5 derived antibodies include CDRs of the heavy chain variable domain, SEQ ID NOs: 4, 5, 6, and CDRs of the light chain variable domain, SEQ ID NOs: 46, 47, 48, as defined by Kabat definition. Under Chothia definition, the CDR sequences of the heavy chain variable domain are set forth in SEQ ID NOs: 25, 26, 27, and CDRs of the light chain variable domain are set forth in SEQ ID NOs: 46, 47, 48.

The CDR sequences for 11C3, and 11C3 derived antibodies include CDRs of the heavy chain variable domain, SEQ ID NOs: 7, 8, 9, and CDRs of the light chain variable domain, SEQ ID NOs: 49, 50, 51, as defined by Kabat definition. Under Chothia definition, the CDR sequences of the heavy chain variable domain are set forth in SEQ ID NOs: 28, 29, 30, and CDRs of the light chain variable domain are set forth in SEQ ID NOs: 49, 50, 51.

The CDR sequences for 11C12, and 11C12 derived antibodies include CDRs of the heavy chain variable domain, SEQ ID NOs: 10, 11, 12, and CDRs of the light chain variable domain, SEQ ID NOs: 49, 50, 51, as defined by Kabat definition. Under Chothia definition, the CDR sequences of the heavy chain variable domain are set forth in SEQ ID NOs: 31, 32, 33, and CDRs of the light chain variable domain are set forth in SEQ ID NOs: 49, 50, 51.

The CDR sequences for 11D2, and I1D2 derived antibodies include CDRs of the heavy chain variable domain, SEQ ID NOs: 13, 14, 15, and CDRs of the light chain variable domain, SEQ ID NOs: 49, 50, 51, as defined by Kabat definition. Under Chothia definition, the CDR sequences of the heavy chain variable domain are set forth in SEQ ID NOs: 34, 35, 36, and CDRs of the light chain variable domain are set forth in SEQ ID NOs: 49, 50, 51.

The CDR sequences for 13F9, and 13F9 derived antibodies include CDRs of the heavy chain variable domain, SEQ ID NOs: 16, 17, 18, and CDRs of the light chain variable domain, SEQ ID NOs: 49, 50, 51, as defined by Kabat definition. Under Chothia definition, the CDR sequences of the heavy chain variable domain are set forth in SEQ ID NOs: 37, 38, 39, and CDRs of the light chain variable domain are set forth in SEQ ID NOs: 49, 50, 51.

The CDR sequences for 14C2, and 14C2 derived antibodies include CDRs of the heavy chain variable domain, SEQ ID NOs: 19, 20, 21, and CDRs of the light chain variable domain, SEQ ID NOs: 49, 50, 51, as defined by Kabat definition. Under Chothia definition, the CDR sequences of the heavy chain variable domain are set forth in SEQ ID NOs: 40, 41, 42, and CDRs of the light chain variable domain are set forth in SEQ ID NOs: 49, 50, 51.

The amino acid sequence for the heavy chain variable region of 5D9 antibody is set forth in SEQ ID NO: 52. The amino acid sequence for the light chain variable region of 5D9 antibody is set forth in SEQ ID NO: 53.

The amino acid sequence for the heavy chain variable region of 7B5 antibody is set forth in SEQ ID NO: 54. The amino acid sequence for the light chain variable region of 7B5 antibody is set forth in SEQ ID NO: 55.

The amino acid sequence for the heavy chain variable region of 11C3 antibody is set forth in SEQ ID NO: 56. The amino acid sequence for the light chain variable region of 11C3 antibody is set forth in SEQ ID NO: 61.

The amino acid sequence for the heavy chain variable region of 11C12 antibody is set forth in SEQ ID NO: 57. The amino acid sequence for the light chain variable region of 11C12 antibody is set forth in SEQ ID NO: 61.

The amino acid sequence for the heavy chain variable region of 11D2 antibody is set forth in SEQ ID NO: 58. The amino acid sequence for the light chain variable region of 11D2 antibody is set forth in SEQ ID NO: 61.

The amino acid sequence for the heavy chain variable region of 13F9 antibody is set forth in SEQ ID NO: 59. The amino acid sequence for the light chain variable region of 13F9 antibody is set forth in SEQ ID NO: 61.

The amino acid sequence for the heavy chain variable region of 14C2 antibody is set forth in SEQ ID NO: 60. The amino acid sequence for the light chain variable region of 14C2 antibody is set forth in SEQ ID NO: 61.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 52, 54, 56, 57, 58, 59, or 60. In some embodiments, the light chain variable region is at least 80%, 85%, 90%, 91%, 92%, 93%, 94%, 95%, 96%, 97%, 98%, or 99% identical to SEQ ID NO: 53, 55, or 61. The heavy chain variable region sequence can be paired with the corresponding light chain variable region sequence, and together they bind to IL2RA.

In some embodiments, the variable regions are fully human, e.g., derived from human heavy chain immunoglobulin locus sequences (e.g., recombination of human IGHV, IGHD, and IGHJ genes), and/or human kappa chain immunoglobulin locus sequences (e.g., recombination of human IGKV and IGKJ genes).

Furthermore, in some embodiments, the antibodies or antigen-binding fragments thereof described herein can also contain one, two, or three heavy chain variable region CDRs selected from the group of SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24, SEQ ID NOs: 25-27, SEQ ID NOs: 28-30, SEQ ID NOs: 31-33, SEQ ID NOs: 34-36, SEQ ID NOs: 37-39, and SEQ ID NOs: 40-42; and/or one, two, or three light chain variable region CDRs selected from the group of SEQ ID NOs: 43-45, SEQ ID NOs: 46-48, and SEQ ID NOs: 49-51.

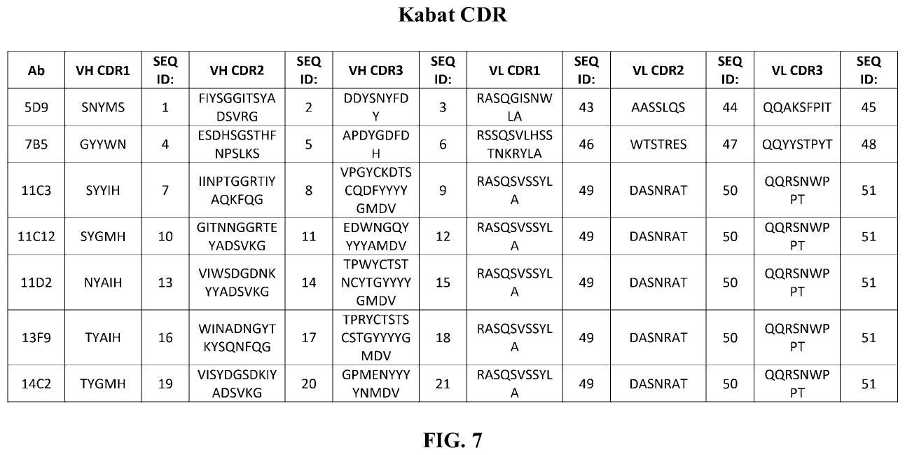

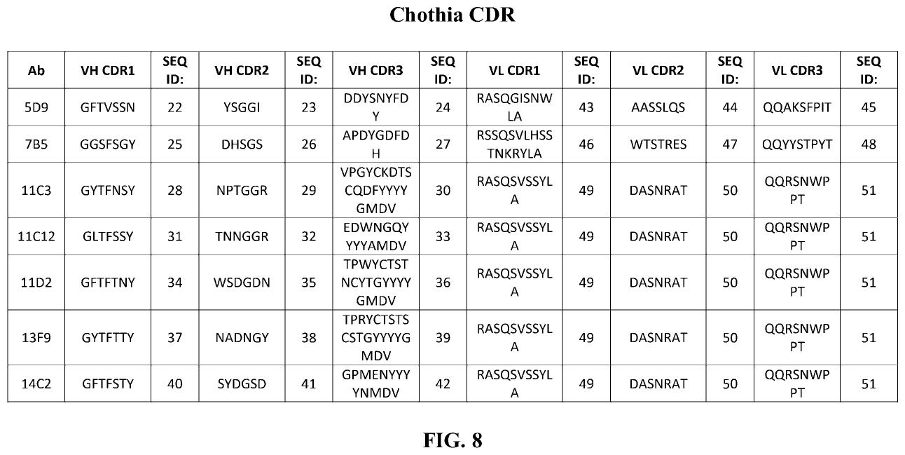

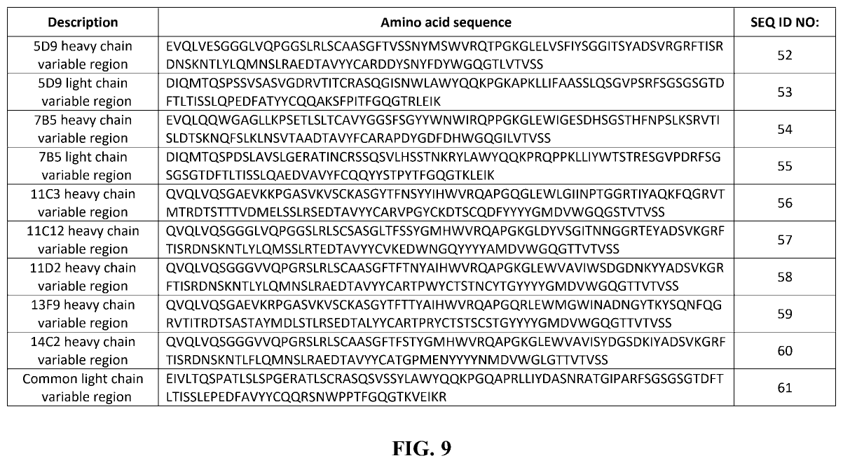

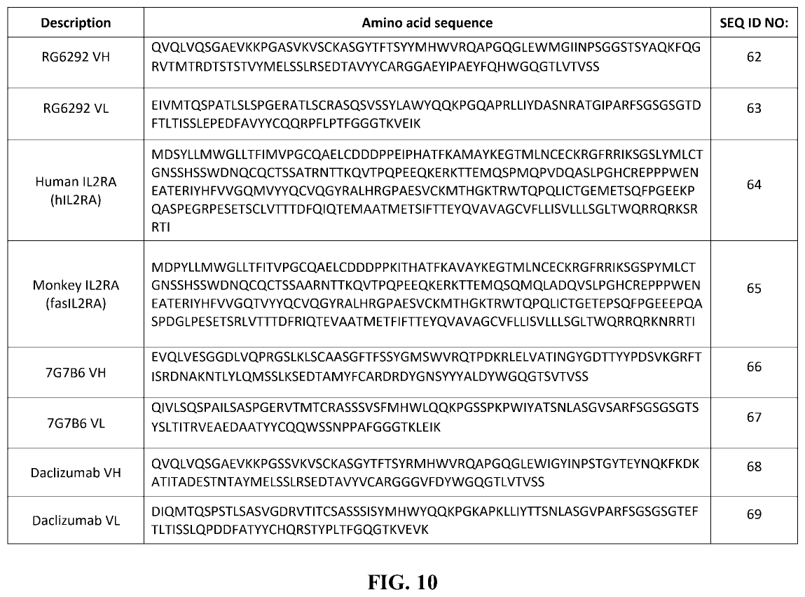

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 7 (CDRs under Kabat definition), and FIG. 8 (CDRs under Chothia definition).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the antibody or an antigen-binding fragment described herein can contain a light chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the antibody or an antigen-binding fragment described herein can contain a light chain variable domain containing one, two, or three of the CDRs of SEQ ID NO: 46 with zero, one or two amino acid insertions, deletions, or substitutions; SEQ ID NO: 47 with zero, one or two amino acid insertions, deletions, or substitutions; SEQ ID NO: 48 with zero, one or two amino acid insertions, deletions, or substitutions.

In some embodiments, the antibody or an antigen-binding fragment described herein can contain a light chain variable domain containing one, two, or three of the CDRs of SEQ ID NO: 49 with zero, one or two amino acid insertions, deletions, or substitutions; SEQ ID NO: 50 with zero, one or two amino acid insertions, deletions, or substitutions; SEQ ID NO: 51 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition scheme. In some embodiments, the CDR is determined based on Chothia definition scheme. In some embodiments, the CDR is determined based on a combination of Kabat and Chothia definition scheme. In some embodiments, the CDR is determined based on IMGT definition. In some embodiments, the CDR is determined based on contact definition.

The disclosure also provides antibodies or antigen-binding fragments thereof that bind to IL2RA.

The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 52, and the selected VL sequence is SEQ ID NO: 53. In some embodiments, the selected VH sequence is SEQ ID NO: 54 and the selected VL sequence is SEQ ID NO: 55. In some embodiments, the selected VH sequence is SEQ ID NO: 56 and the selected VL sequence is SEQ ID NO: 61. In some embodiments, the selected VH sequence is SEQ ID NO: 57 and the selected VL sequence is SEQ ID NO: 61. In some embodiments, the selected VH sequence is SEQ ID NO: 58 and the selected VL sequence is SEQ ID NO: 61. In some embodiments, the selected VH sequence is SEQ ID NO: 59 and the selected VL sequence is SEQ ID NO: 61. In some embodiments, the selected VH sequence is SEQ ID NO: 60 and the selected VL sequence is SEQ ID NO: 61.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 7, or FIG. 8, or have sequences as shown in FIG. 9. When the polypeptides are paired with corresponding polypeptide (e.g., a corresponding heavy chain variable region or a corresponding light chain variable region), the paired polypeptides bind to IL2RA.

The anti-IL2RA antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multimeric, multi-specific (e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgGl, IgG2, IgG3, IgG4, IgAQ1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to IL2RA will retain an ability to bind to IL2RA. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides various antibodies and antigen-binding fragments thereof derived from anti-IL2RA antibodies described herein. In general, antibodies (also called immunoglobulins) are made up of two classes of polypeptide chains, light chains and heavy chains. A non-limiting examples of antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgGl, IgG2, IgG2a, IgG2b, IgG3, IgG4, IgEl, IgE2, etc.

The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the antigen binding surface of the antibody. The four framework regions largely adopt a beta-sheet conformation and the CDRs form loops connecting the beta-sheet structure, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et at., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

Fragments of antibodies are suitable for use in the methods described herein are also provided. The Fab fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fe region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, teniposide, vincristine, vinblastine, colchicin, doxorubicin, daunorubicin, dihydroxyanthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting IL2RA and an addition antigen (e.g., OX40, CD3, 4-1BB, CD314, CD47, PD-1, CTLA4, CD40 or PDL1).

### Antibody Characteristics

In some embodiments, the antibodies or antigen-binding fragments thereof described herein cannot block the binding between IL2RA and IL2RA ligands (e.g., IL2). Thus, by binding to IL2RA, the antibody does not inhibit IL2RA signaling pathway. In some embodiments, the antibody can upregulate immune response. In some embodiments, the antibody can reduce tumor volume in an animal.

In some implementations, the antibody (or antigen-binding fragments thereof) specifically binds to IL2RA (e.g., human IL2RA or monkey IL2RA) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity of an antibody for an antigen include, e.g., ELISA, RIA, and surface plasmon resonance (SPR). In some embodiments, the antibody binds to human IL2RA (SEQ ID NO: 64), and/or monkey IL2RA (e.g., SEQ ID NO: 65). In some embodiments, the antibody does not bind to human IL2RA and/or monkey IL2RA.

In some embodiments, the antibody or antigen-binding fragment thereof described herein does not inhibit IL2-induced STATS phosphorylation, or inhibit STAT5 phosphorylation by less than 20%, less than 15%, less than 10%, less than 5%, or less than 1%.

In some embodiments, the antibody or antigen-binding fragment thereof described herein has a tumor growth inhibition percentage (TGI_{TV}%) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI(%)=[1−(Tᵢ−T₀)/(Vᵢ−V₀)]×100%

Tᵢ is the average tumor volume in the treatment group on Day i. T₀ is the average tumor volume in the treatment group on Day zero. Vᵢ is the average tumor volume in the control group on Day i. V₀ is the average tumor volume in the control group on Day zero.

In some embodiments, the antibodies or antigen-binding fragments thereof as described herein are IL2RA antagonist. In some embodiments, the antibodies or antigen-binding fragments thereof as described herein are IL2RA agonist.

In some embodiments, the antibodies or antigen-binding fragments thereof as described herein are not toxic. In some embodiments, no significant differences in body weight can be observed between treatment group and control group e.g., at 0.3 mg/kg, 1 mg/kg, 10 mg/kg, or 25 mg/kg.

In some embodiments, the antibodies or antigen binding fragments can induce complement-dependent cytotoxicity (CDC) and/or antibody dependent cellular cytotoxicity (ADCC), and kill the tumor cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fe region is phagocytosis. In some embodiments, effector function of a functional Fc region is ADCC and phagocytosis.

In some embodiments, the antibodies or antigen binding fragments can induce complement-dependent cytotoxicity (CDC).

In some embodiments, the Fe region is human IgG1, human IgG2, human IgG3, or human IgG4. In some embodiments, the antibody is a human IgG1 antibody.

In some embodiments, the antibodies or antigen binding fragments do not have a functional Fc region. For example, the antibodies or antigen binding fragments are Fab, Fab′, F(ab′)₂, and Fv fragments. In some embodiments, the Fc region has LALA mutations (L234A and L235A mutations in EU numbering), or LALA-PG mutations (L234A, L235A, P329G mutations in EU numbering).

In some embodiments, the Fc have a SI mutation (S239D and I332E mutations in EU numbering).

### Methods of Making Anti-IL2RA Antibodies

An isolated fragment of human IL2RA (e.g., extracellular region) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of IL2RA and encompasses an epitope of the protein such that an antibody raised against the peptide forms a specific immune complex with the protein. As described above, the full length sequence of human TL2RA is known in the art (SEQ ID NO: 64). In some embodiments, an Fc-tagged human IL2RA protein (the Fc fusion protein contains all or a portion of human IL2RA extracellular domain, e.g., positions 22-213 of SEQ ID NO: 64) is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human IL2RA). The preparation can further include an adjuvant, such as Freund's complete or incomplete adjuvant, or a similar immunostimulatory agent.

Polyclonal antibodies can be prepared as described above by immunizing a suitable subject with a IL2RA polypeptide, or an antigenic peptide thereof (e.g., part of IL2RA, such as the extracellular region) as an immunogen. The antibody titer in the immunized subject can be monitored over time by standard techniques, such as with an enzyme-linked immunosorbent assay (ELISA) using the immobilized IL2RA polypeptide or peptide. If desired, the antibody molecules can be isolated from the mammal (e.g., from the blood) and further purified by well-known techniques, such as protein A or protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., IL2RA. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al. “Replacing the complementarity-determining regions in a human antibody with those from a mouse.” Nature 321.6069 (1986): 522; Riechmann et al. “Reshaping human antibodies for therapy.” Nature 332.6162 (1988): 323; Dall'Acqua et al. “Antibody humanization by framework shuffling.” Methods 36.1 (2005): 43-60; each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al. “A humanized CD18 antibody can block function without cell destruction.” The Journal of Immunology 151.4 (1993): 2296-2308; Chothia, et al., “Canonical structures for the hypervariable regions of immunoglobulins.” Journal of Molecular Biology 196.4 (1987): 901-917).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-IL2RA antibody will contain an amino acid sequence having at least 75%, 80%, 85%, 90%, 95%, 96%, 97%, 98%, or 99% percent identity with a sequence present in the tight or heavy chain of the original antibody.

In some embodiments, a mouse (e.g., RenMab mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab mice can be found in PCT/CN2020/075698, which is incorporated herein by reference in its entirety. The antibodies generated by the mice have a full human VH, a full human VL, and mouse constant regions. In some embodiments, the human VH and human VL is linked to a human IgG constant regions (e.g., IgGl, IgG2, IgG3, and IgG4).

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chains. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

Identity or homology with respect to an original sequence is usually the percentage of amino acid residues present within the candidate sequence that are identical with a sequence present within the human, humanized, or chimeric anti-IL2RA antibody or fragment, after aligning the sequences and introducing gaps, if necessary, to achieve the maximum percent sequence identity, and not considering any conservative substitutions as part of the sequence identity.

Additional modifications to the anti-IL2RA antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fe regions.

In some embodiments, a covalent modification can be made to the anti-IL2RA antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fe region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. NatI. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-1 promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, neurologic cancer, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, skin cancer or hematologic malignancy. In some embodiments, the cancer is unresectable melanoma or metastatic melanoma, non-small cell lung carcinoma (NSCLC), small cell lung cancer (SCLC), bladder cancer, gastroesophageal junction cancer or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, glioma, hematological malignancies, especially Non-Hodgkin's lymphoma, lymphoma, chronic lymphocytic leukemia, or advanced solid tumors.

In some embodiments, the anti-IL2RA antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the anti-IL2RA antibody is designed for treating hepatocellular, ovarian, colon, or cervical carcinomas. In some embodiments, the anti-IL2RA antibody is designed for treating advanced breast cancer, advanced ovarian cancer, and/or advanced refractory solid tumor. In some embodiments, the anti-IL2RA antibody is designed for treating metastatic solid tumors, NSCLC, melanoma, non-Hodgkin lymphoma, colorectal cancer, and multiple myeloma. In some embodiments, the anti-IL2RA antibody is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the anti-IL2RA antibody is designed for treating carcinomas (e.g., nasopharynx carcinoma, bladder carcinoma, cervix carcinoma, kidney carcinoma or ovary carcinoma).

In some embodiments, the IL2RA antibody is designed for treating acute lymphoblastic leukemia (ALL), B-cell chronic lymphocytic leukemia (B-CLL), hairy cell leukemia (HCL), solid tumors, colorectal cancer, ovarian cancer, prostate cancer, melanoma, lung cancer, breast cancer, gastric cancer, esophageal squamous cell carcinoma (ESCC), leukemia, lymphoma, multiple myeloma, sarcoma, and/or head-and-neck cancer.

In some embodiments, the compositions and methods disclosed herein can be used for treatment of patients at risk for a cancer. Patients with cancer can be identified with various methods known in the art.

In some aspects, the disclosure relates to a method of treating an autoimmune disease or inflammation, the method comprising administering to the subject an effective amount of a composition comprising the antibody or antigen-binding fragment thereof as described herein or the antibody-drug conjugate as described herein.

In one aspect, the disclosure provides methods for treating, preventing, or reducing the risk of developing disorders associated with an abnormal or unwanted immune response, e.g., an autoimmune disorder. These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-IL2RA antibodies or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has Crohn's disease, ulcerative colitis or type 1 diabetes. In some embodiments, the antibodies or antigen binding fragments can be used to treat inflammation. In some embodiments, the anti-IL2RA antibody is designed for treating other diseases or disorders, e.g., microbial infection and allergic disorders.

In some embodiments, the antibody or antigen binding fragment thereof described herein can specifically target Treg cells without blocking the interaction between IL2 and IL2RA. As a result, they can be used to eliminate Treg cells thereby relieving immunosuppression. Meanwhile, the preservation of the IL2/IL2RA interaction allows effective stimulation of T cells by activating the IL2/IL2RA signaling pathway.

In some aspects, the disclosure relates to a method of inhibiting immune response in a subject, the method comprising administering to the subject an effective amount of a composition comprising the antibody or antigen-binding fragment thereof as described herein or the antibody-drug conjugate as described herein. In some embodiments, the subject has an autoimmune disease.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal.

A typical daily dosage of an effective amount of an antibody is 0.01 mg/kg to 100 mg/kg (mg per kg of patient weight). In some embodiments, the dosage can be less than 100 mg/kg, 50 mg/kg, 40 mg/kg, 30 mg/kg, 25 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.3 mg/kg, or 0.1 mg/kg. In some embodiments, the dosage can be greater than 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.3 mg/kg, 0.1 mg/kg, 0.05 mg/kg, or 0.01 mg/kg. In some embodiments, the dosage is about 50 mg/kg, 40 mg/kg, 30 mg/kg, 25 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDOl) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, IL-1, HMGB1, an IL-10 antagonist, an IL-4 antagonist, an IL-6 antagonist (e.g., IL-6 receptor), an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-Li antibody, an anti-PD-L2 antibody, an anti-LAG-3 antibody, an anti-TIGIT antibody, an anti-BTLA antibody, an anti-CTLA-4 antibody, an anti-GITR antibody, an anti-TIM-3 antibody, an anti-4-1BB antibody or an anti-CD40 antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 0.3 mg/kg to about 25 mg/kg, about 1 mg/kg to about 10 mg/kg; or about 1 mg/kg to about 5 mg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Anti-IL2RA Antibody

To generate antibodies against human IL2RA, RenMice (i.e., an engineered mouse comprising DNA encoding human immunoglobulin heavy and kappa light chain variable regions, for example, RenMab™ mice, RenLite™ mice) were immunized with Fc-tagged human IL2RA protein (ACROBiosystems Inc., Cat #: ILA-H5251) or plasmids encoding IL2RA protein. The antibody immune response was monitored by an antigen-specific immunoassay.

A total of 3 immunizations were performed. The immunizations were separated by two weeks. One week after the last immunization, retro-orbital blood was collected, and the antibody titer in serum was determined by Fluorescence-Activated Cell Sorting (FACS). Mice with high titer were selected two weeks later for impulse immunizations. Human IL2RA protein or CHO-S cells expressing human IL2RA protein were used for impulse immunizations by intraperitoneal injection and tail vein injection, respectively.

When a desired immune response was achieved, antigen-specific immune cells were isolated from the immunized mice to further obtain anti-IL2RA antibodies or to obtain the light chain and heavy chain variable region sequences of the anti-IL2RA antibodies. For example, single cell technology (for example, using Beacon® Optofluidic System, Berkeley Lights Inc.) was used to screen and find plasma cells that secrete antigen-specific monoclonal antibodies, and reverse transcription and PCR sequencing were used to obtain antibody variable region sequences. The obtained variable region sequences were cloned into a vector containing a sequence encoding the human IgG constant region for antibody expression. The binding affinity of the expressed antibody to IL2RA was verified using FACS.

Exemplary antibodies obtained included: 5D9, 7B5, 11C3, 1IC12, 11D2, 13F9 and 14C2. The heavy and light chain variable regions of 5D9, 7B5, 11C3, 11C12, 11D2, 13F9 and 14C2 are shown in FIG. 9. FIG. 7 shows the CDR sequences according to Kabat definition. FIG. 8 shows the CDR sequences according to Chothia definition.

Various IgG1, IgG2 and IgG4 antibodies were made. With respect to the name of the antibodies, when the antibody VH/VL is connected to different isotypes, the isotype is added to the name. For example, if the VH and VL of 5D9 are connected to IgG1 constant regions, the antibody is named as 5D9-IgG1 (or 5D9). Examples of other isotypes are as follows: 5D9-IgG2 and 5D9-IgG4. The constant region of the antibodies can include some mutations. For example, when SI mutations (EU numbering: S239D and I332E mutations) are introduced into the Fc region of 5D9-IgG1, the resulting antibody is named as 5D9-IgG1-SI (or 5D9-SI).

### Example 2. Binding Affinity of Anti-IL2RA Antibodies

The affinity of the anti-IL2RA antibodies to His-tagged human IL2RA protein (hIL2RA-his, ACROBiosystems Inc., Cat #: ILA-H52H9) and His-tagged recombinant monkey (Cynomolgus monkey, or Macacafascicularis) IL2RA protein (cynoIL2RA-his, Sino Biological, Inc., Cat #: 90265-C08H) were measured by surface plasmon resonance (SPR) using Biacore™ (Biacore, INC, Piscataway N.J.) 8K biosensor equipped with pre-immobilized Protein A sensor chips.

Purified anti-IL2RA antibodies were diluted to 2 g/mL and then injected into the Biacore™ 8K biosensor at 10 L/min for about 50 seconds to achieve a desired protein density (e.g., about 150 response units (RU)). The His-tagged IL2RA protein at a concentration of 200, 100, 50, 25, 12.5, 6.25, 3.125 or 0 nM was then injected at 30 μL/min for 180 seconds. Dissociation was monitored for 400 seconds. The chip was regenerated after the last injection of each titration with glycine (pH 2.0, 30 L/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below. ISO is an antibody with the same IgG1 subtype but targeting an unrelated target.

TABLE 1
Affinity test results

| Antibody | Protein | kon (1/Ms) | koff (1/s) | KD (M) |
|---|---|---|---|---|
| RG6292 analog | hIL2RA-his | 4.63E+04 | 2.03E−03 | 4.38E−08 |
| | cynoIL2RA-his | — | — | — |
| 5D9 | hIL2RA-his | 8.38E+04 | 4.30E−04 | 5.14E−09 |
| | cynoIL2RA-his | 7.14E+04 | 1.93E−02 | 2.71E−07 |
| 7B5 | hIL2RA-his | 2.25E+06 | 1.43E−02 | 6.35E−09 |
| | cynoIL2RA-his | 1.05E+06 | 2.58E−02 | 2.46E−08 |
| 11C3 | hIL2RA-his | 3.54E+05 | 9.42E−05 | 2.66E−10 |
| | cynoIL2RA-his | 3.14E+05 | 1.66E−04 | 5.28E−10 |
| 11C12 | hIL2RA-his | 3.01E+05 | 2.68E−03 | 8.90E−09 |
| | cynoIL2RA-his | 2.91E+05 | 3.43E−02 | 1.18E−07 |
| 11D2 | hIL2RA-his | 1.85E+05 | 2.35E−04 | 1.27E−09 |
| | cynoIL2RA-his | 3.56E+05 | 9.01E−04 | 2.53E−09 |
| 13F9 | hIL2RA-his | 3.23E+04 | 2.11E−05 | 6.55E−10 |
| | cynoIL2RA-his | 3.78E+04 | 4.47E−04 | 1.18E−08 |
| 14C2 | hIL2RA-his | 2.58E+05 | 9.62E−04 | 3.72E−09 |
| | cynoIL2RA-his | 2.18E+05 | 1.16E−02 | 5.31E−08 |
| ISO | hIL2RA-his | — | — | — |
| | cynoIL2RA-his | — | — | — |

(“—” means no binding)

RG6292 is a human IgG1 monoclonal antibody targeting IL2RA in early clinical development at Roche for the treatment of patients with advanced and/or metastatic solid tumors, and its VH and VL sequences are shown in SEQ ID NO: 62 and SEQ ID NO: 63, respectively.

The results showed that the anti-IL2RA antibodies 5D9, 7B5, 11C3, 11C12, 11D2, 13F9 and 14C2 had good binding affinities to human IL2RA and monkey IL2RA.

### Example 3. Binding Activity of Anti-IL2RA Antibodies

The binding activity of anti-IL2RA antibodies (5D9, 7B5, 11C3, 11C12, 11D2, 13F9 and 14C2) to CHO-S-hIL2RA cells (CHO-S cells expressing human IL2RA (hIL2RA, SEQ ID NO: 64)) or CHO-S-fas IL2RA cells (CHO-S cells expressing monkey (Macaca fascicularis) IL2RA (fasIL2RA, SEQ ID NO: 65)) was verified by flow cytometry.

CHO-S-hIL2RA cells or CHO-S-fasIL2RA cells were plated in a 96-well plate at a density of 2×10⁵ cells/well. Serially diluted purified anti-IL2RA antibodies (maximum concentration: 9 μg/mL, 2-fold dilutions, 11 gradients) were added to each well and were incubated at 4° C. for 30 minutes. Then, after one wash with PBS, the cells were incubated with the secondary antibody Alexa Fluor® 647 anti-human IgG Fcγ (Jackson ImmunoResearch Laboratories, Inc., Cat #:109-606-170) at 4° C. for 15 minutes before flow cytometry analysis.

The cells were collected, and the mean fluorescence intensity (MFI) was determined. A fitting curve was obtained using Log(antibody concentration (μg/mL)) as the X-axis and mean MFI as the Y-axis. EC50 values were determined and the test results are shown in the table below.

TABLE 2
Binding of the antibodies to cells

EC50 (μg/mL)

| Antibody | CHO-S-hIL2RA cells | CHO-S-fasIL2RA cells |
|---|---|---|
| 5D9 | 0.470 | 0.466 |
| 7B5 | 0.411 | 0.435 |
| 11C3 | 0.099 | 0.111 |
| 11C12 | 0.083 | 0.091 |
| 11D2 | 0.052 | 0.039 |
| 13F9 | 0.326 | 0.275 |
| 14C2 | 0.093 | 0.088 |

The results showed that all seven antibodies 5D9, 7B5, 11C3, 11C12, 11D2, 13F9 and 14C2 had good binding activity to human IL2RA and monkey IL2RA.

### Example 4. Blockade Assays

### Blockade Assays of the Binding of IL2RA Ligand and IL2RA by Flow Cytometry

Blockade of human IL2RA ligand (Biotinylated Human IL-2 Protein, His, Avitag, ACROBiosystems Inc., Cat #: IL2-H82E4) binding to human IL2RA by anti-IL2RA antibodies were tested by flow cytometry using human CHO-S-hIL2RA cells.

CHO-S-hIL2RA cells were seeded in a 96-well plate (cell density 2×10⁵ cells/well) and incubated at 37° C. overnight. Serially diluted tested antibodies (2-fold diluted with the highest concentration at 20 μg/mL, 11 gradients) were incubated with the cells at 4° C. for 15 minutes, and then human IL2RA ligand was added to each well. The working concentration of the human IL2RA ligand was 2.5 μg/mL. The above 96-well plate was incubated at 4° C. for 20 minutes. Then, after one wash with PBS, the cells were incubated with the secondary antibody APC Streptavidin (BioLegend, Cat #: 405207) at 4° C. for 15 minutes before flow cytometry analysis.

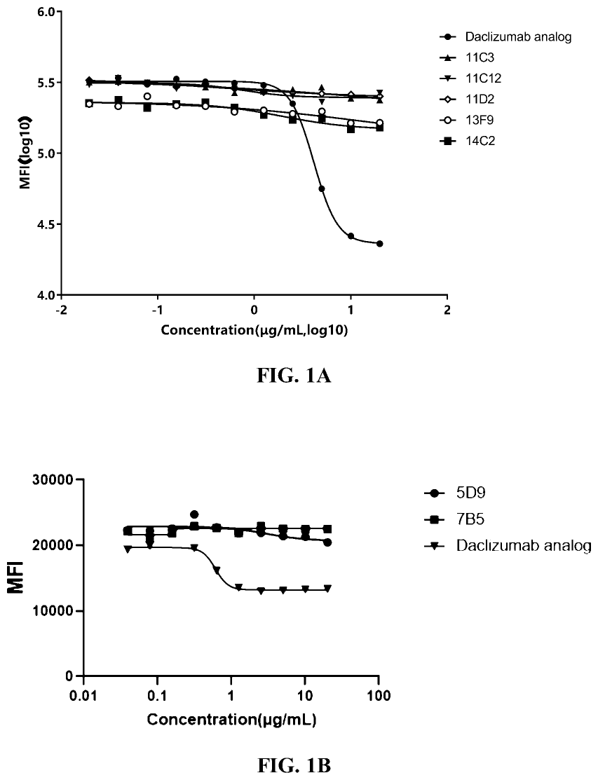

The mean fluorescence intensity (MFI) was determined. A fitting curve was obtained using Log(antibody concentration (μg/mL)) or antibody concentration (μg/mL) as the X-axis and Log(mean MFI) or MFI as the Y-axis. The results are shown in FIGS. 1A-1B.

Daclizumab is a humanized monoclonal antibody that binds to the IL-2 receptor (alpha subunit, CD25) on activated T-cells and inhibits IL-2 mediated activation of lymphocytes, preventing the activation of the inflammatory cytokine response common to transplant rejection and autoimmune and inflammatory diseases, and its VH and VL sequences are shown in SEQ ID NO: 68 and SEQ ID NO: 69, respectively.

When the concentration of antibodies (5D9, 7B5, 11C3, 1IC12, 11D2, 13F9 and 14C2) increased, the MFI didn't decreased accordingly, suggesting that the anti-IL2RA antibodies didn't block the binding of human IL2RA to IL2RA ligand, while Daclizumab analog blocked the binding of human IL2RA and IL2RA ligand.

### IL2 Reporter Cell Blockade Assays

The experiment was performed to test whether anti-IL2RA antibodies 5D9, 7B5, 11C3, 11C12, 11D2, 13F9 and 14C2 can block the binding of mouse IL2 protein (mIL2) and IL2 reporter cells.

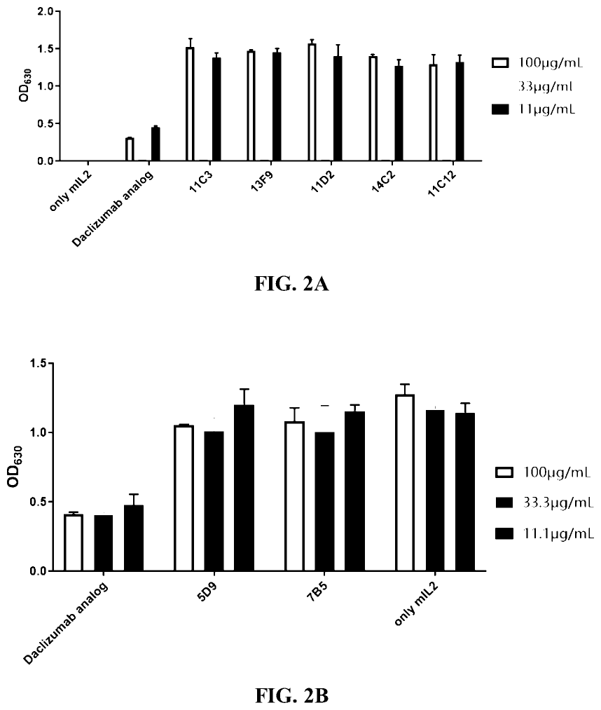

20 μL diluted mouse IL2 protein (mIL2, ACRO, Cat #:IL2-M52H3) (100 ng/mL) were added in a 96-well plate. 20 μL of the diluted antibody solution were then added to each well. The working concentration of the diluted antibody was 100 μg/mL, 33.3 μg/mL or 11.1 μg/mL. Then, 160 μL IL2 reporter cells (HEK293-Blue-IL2 cells, Invivogen, Cat #: hkb-il2) were seeded in the 96-well plate (cell density 5×10⁴ cells/well) and the above 96-well plate was incubated at 37° C., 5% CO₂ for 20-24 hours. After the incubation, the plate was taken out, and 180 μL QUANTI-Blue™ Solution (QUANTI Blue reagent:QUANTI Blue buffer:Sterile water=1:1:98) and 20 μL cell supernatant were added to each well. The plate was incubated at 37° C. for 1 hour, and then placed in a microplate reader to detect the OD value at 630 nm. The results are shown in FIGS. 2A-2B.

The results showed that the anti-IL2RA antibodies 5D9, 7B5, 11C3, 11C12, 11D2, 13F9 and 14C2 didn't block the binding of mouse IL2 protein and IL2 reporter cells, while Daclizumab analog blocked the binding of mouse IL2 protein and IL2 reporter cells.

### STAT5 Phosphorylation Blocking Activity Detection

Human CD3⁺ T cells were incubated at 37° C. for 2 hours and then seeded in a 96-well plate (100 μL/well). Antibodies were diluted and added to the wells and incubated with the cells at 37° C., 5% CO₂ for 30 minutes (the working concentration of the diluted antibody was 10 μg/mL). Human IL2 protein (hIL2, ACROBiosystems Inc., Cat #: IL2-H4113) (5 U/mL) was then added to each well and the above 96-well plate was incubated at 37° C., 5% CO₂ for 15 minutes. Then, the cells were collected and a single-cell suspension was prepared from the samples. The cells were labeled with PE anti-STAT5 Phospho (Tyr694) Antibody (BioLegend, Cat #: 936904), followed by flow cytometry analysis.

7G7B6 is a mouse IgG2 monoclonal antibody targeting human IL2RA in preclinical development at Leukemia Therapy, and its VH and VL sequences are shown in SEQ ID NO: 66 and SEQ lID NO: 67, respectively.

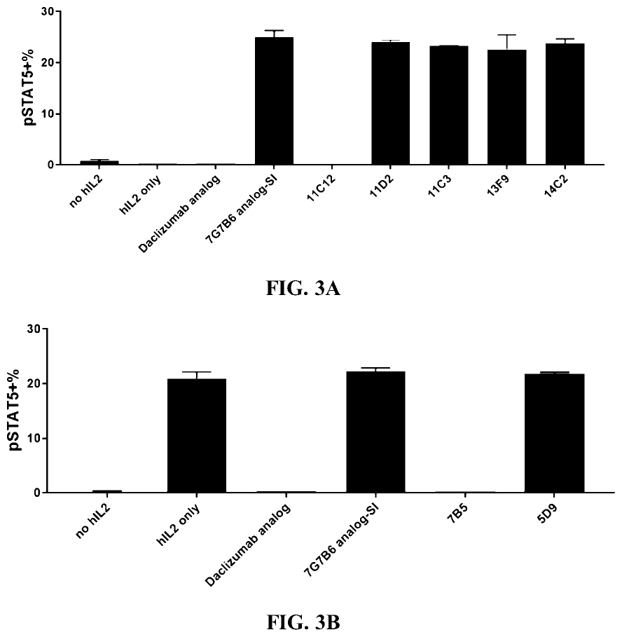

The results are shown in FIGS. 3A-3B. Similar to 7G7B6 analog-SI, anti-IL2RA antibodies 5D9, 7B5, 11C3, 11C12, 11D2, 13F9 and 14C2 exhibited little effect on human IL2-induced STAT5 phosphorylation, indicating that they are pSTAT5 non-blocking antibodies. Daclizumab analog can significantly inhibit STAT5 phosphorylation, thus it is a pSTAT5 blocking antibody.

### Example 5. Anti-Tumor Activity in MC38 Xenograft Model

### Anti-Tumor Activity of Anti-IL2RA Antibodies 7B5 and 5D9

Anti-IL2RA antibodies were tested for their effects on tumor growth in vivo in a colon cancer model. About 5×10⁵ MC38 cells (mouse colon cancer cells) were injected subcutaneously in each B-hIL2RA mouse (Biocytogen Pharmaceuticals (Beijing) Co., Ltd., Cat #: 110066). When the tumors in the mice reached a volume of about 100 mm³, the mice were randomly placed into different groups based on tumor size. The mice were then injected with phosphate buffer saline (PBS) or anti-IL2RA antibodies by intraperitoneal (i.p.) administration. Details are shown in the table below.

TABLE 3
Group assignment

| | No. of | | | | | | Total No. of |
|---|---|---|---|---|---|---|---|
| Group | mice | Antibody | Dosage | | Route | Frequency | administration |
| G1 | 6 | PBS | — | | i.p. | BIW | 6 |
| G2 | 6 | 7B5 | 3 | mg/kg | i.p. | BIW | 6 |
| G3 | 6 | 7B5 | 10 | mg/kg | i.p. | BIW | 6 |
| G4 | 6 | 5D9 | 3 | mg/kg | i.p. | BIW | 6 |
| G5 | 6 | 5D9 | 10 | mg/kg | i.p. | BIW | 6 |
| G6 | 6 | 7G7B6 | 3 | mg/kg | i.p. | BIW | 6 |
| | | analog | | | | | |
| G7 | 6 | 7G7B6 | 10 | mg/kg | i.p. | BIW | 6 |
| | | analog | | | | | |

The length of the long axis and the short axis of the tumor were measured and the volume of the tumor was calculated as 0.5×(long axis)×(short axis)².

The tumor growth inhibition percentage (TGI %) was calculated using the following formula: TGI (%)=[1−(Tᵢ−T₀)/(Vᵢ−V₀)]×100%. Tᵢ is the average tumor volume in the treatment group on day i. Tₒ is the average tumor volume in the treatment group on Day 0. Vᵢ is the average tumor volume in the control group on Day i. V₀ is the average tumor volume in the control group on Day 0.

Values are expressed as mean±SEM. T-test was performed for statistical analysis. P<0.05 is a threshold to indicate a significant difference.

The weights of mice in different groups all increased. On the day of group assignment (Day 0), the average weight of each group was in the range of 21.2 g-21.5 g. At the end of the experiment (Day 21), the average body weight of each group was in the range of 24.6 g-25.6 g and the average body weight change of each group was in the range of 115.4%-119.8%. The results showed that these anti-IL2RA antibodies were well tolerated and were not obviously toxic to the mice.

The table below summarizes the results for this experiment, including the tumor volumes on the day of grouping (Day 0), 11 days after grouping (Day 11) and 21 days after grouping (Day 21); Tumor Growth Inhibition value (TGI); and the statistical differences (P value) of tumor volume and body weight between the treatment and control groups.

TABLE 4
Tumor size changes

| | | | | | P value | |
|---|---|---|---|---|---|---|
| | Tumor volume (mm³) | | | | Body | Tumor |
| Group | Day 0 | Day 11 | Day 21 | TGI (%) | weight | volume |
| G1 | 98 ± 4 | 1092 ± 93 | 2559 ± 349 | NA | NA | NA |
| G2 | 98 ± 4 | 591 ± 85 | 1089 ± 283 | 59.7 | 0.683 | 0.008 |
| G3 | 98 ± 5 | 699 ± 98 | 931 ± 207 | 66.1 | 0.722 | 0.002 |
| G4 | 98 ± 5 | 734 ± 144 | 1273 ± 403 | 52.3 | 0.862 | 0.036 |
| G5 | 98 ± 5 | 650 ± 75 | 960 ± 179 | 65.0 | 0.749 | 0.002 |
| G6 | 98 ± 5 | 787 ± 129 | 1729 ± 567 | 33.7 | 0.693 | 0.241 |
| G7 | 98 ± 6 | 859 ± 185 | 1355 ± 400 | 48.9 | 0.938 | 0.047 |

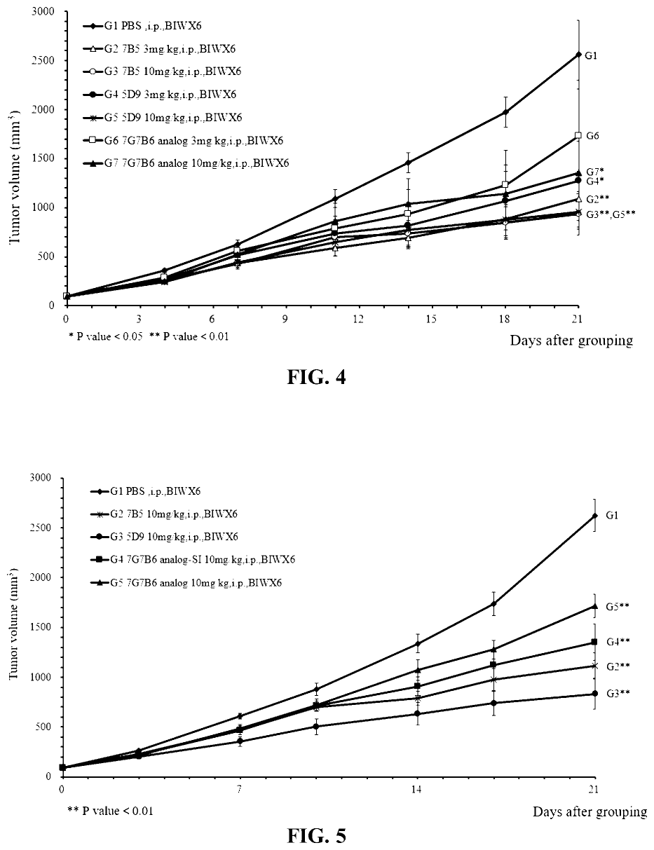

The tumor volume of mice in different groups treated with the antibodies or PBS are shown in FIG. 4. The anti-IL2RA antibodies (G2-G5) showed a better tumor inhibitory effect compared with the positive control 7G7B6 analog (G6-G7) under the same dose levels, in a dose-dependent manner.

In another experiment, about 5×10⁵ MC38 cells were injected subcutaneously in B-hIL2RA mice to determine the anti-tumor activity of 7B5 and 5D9. When the tumor reached a volume of about 80-90 mm³, the mice were randomly placed into a control group and different treatment groups based on tumor size. Details of grouping and dosing are shown in the table below.

TABLE 5
Group assignment

| | No. of | | | | | Total No. of |
|---|---|---|---|---|---|---|
| Group | mice | Antibodies | Dosage | Route | Frequency | administration |
| G1 | 6 | PBS | — | i.p. | BIW | 6 |
| G2 | 6 | 7B5 | 10 mg/kg | i.p. | BIW | 6 |
| G3 | 6 | 5D9 | 10 mg/kg | i.p. | BIW | 6 |
| G4 | 6 | 7G7B6 | 10 mg/kg | i.p. | BIW | 6 |
| | | analog-SI | | | | |
| G5 | 6 | 7G7B6 | 10 mg/kg | i.p | BIW | 6 |
| | | analog | | | | |

The weights of mice in different groups all increased which showed that 7B5 and 5D9 were well tolerated and were not obviously toxic to the mice. The table below summarizes the results of this experiment, including the tumor volumes on the day of grouping (Day 0), 10 days after grouping (Day 10) and at the end of the experiment (Day 21); TGI (%); and the statistical differences (P value) of tumor volume and body weight between the treatment and control groups.

TABLE 6
Tumor size changes

| | | | | | P value | |
|---|---|---|---|---|---|---|
| | Tumor volume (mm³) | | | | Body | Tumor |
| Group | Day 0 | Day 10 | Day 21 | TGI (%) | weight | volume |
| G1 | 90 ± 3 | 880 ± 63 | 2623 ± 164 | NA | NA | NA |
| G2 | 90 ± 4 | 700 ± 44 | 1117 ± 126 | 59.5 | 0.116 | 2.668E−05 |
| G3 | 90 ± 4 | 505 ± 79 | 833 ± 153 | 70.7 | 0.158 | 1.191E−05 |
| G4 | 90 ± 4 | 716 ± 50 | 1353 ± 186 | 50.2 | 0.159 | 4.454E−04 |
| G5 | 90 ± 4 | 719 ± 61 | 1715 ± 119 | 35.8 | 0.361 | 0.001 |

The tumor volume of mice in different groups treated with the antibodies or PBS are shown in FIG. 5, in which the anti-IL2RA antibodies 7B5 and 5D9 showed a better tumor inhibitory effect compared with 7G7B6 analog-SI and 7G7B6 analog at a dose level of 10 mg/kg.

Anti-TumorActivity of anti-IL2RA antibodies 11C3, 11C12, 11D2, 13F9 and 14C2 About 5×10⁵ MC38 cells (mouse colon cancer cells) were injected subcutaneously in B-hIL2RA mice to determine the anti-tumor activity of 11C3, 11C12, 11D2, 13F9 and 14C2. When the tumors in the mice reached a volume of about 80-90 mm³, the mice were randomly placed into different groups based on tumor size. The mice were then injected with PBS or anti-IL2RA antibodies by intraperitoneal (i.p.) administration. Details are shown in the table below.

TABLE 7
Group assignment

| | No. of | | | | | Total No. of |
|---|---|---|---|---|---|---|
| Group | mice | Antibody | Dosage | Route | Frequency | administration |
| G1 | 6 | PBS | — | i.p. | BIW | 6 |
| G2 | 6 | 7G7B6 | 10 mg/kg | i.p | BIW | 6 |
| | | analog-SI | | | | |
| G3 | 6 | 11C3 | 10 mg/kg | i.p. | BIW | 6 |
| G4 | 6 | 13F9 | 10 mg/kg | i.p. | BIW | 6 |
| G5 | 6 | 11D2 | 10 mg/kg | i.p | BIW | 6 |
| G6 | 6 | 11C12 | 10 mg/kg | i.p. | BIW | 6 |
| G7 | 6 | 14C2 | 10 mg/kg | i.p. | BIW | 6 |

The weights of mice in different groups all increased. On the day of group assignment (Day 0), the average weight of each group was in the range of 20.7 g-21.7 g. At the end of the experiment (Day 22), the average body weight of mice in each group was in the range of 23.8 g-24.9 g and the average body weight change of mice in each group was in the range of 113.5%-117.8%. The results showed that these anti-IL2RA antibodies were well tolerated and were not obviously toxic to the mice.

The table below summarizes the results for this experiment, including the tumor volumes on the day of grouping (Day 0), 12 days after grouping (Day 12) and 22 days after grouping (Day 22); Tumor Growth Inhibition value (TGI); and the statistical differences (P value) of tumor volume and body weight between the treatment and control groups.

TABLE 8
Tumor size changes

| | | | | | P value | |
|---|---|---|---|---|---|---|
| | Tumor volume (mm³) | | | | Body | Tumor |
| Group | Day 0 | Day 12 | Day 22 | TGI (%) | weight | volume |
| G1 | 98 ± 3 | 1172 ± 145 | 2929 ± 434 | NA | NA | NA |
| G2 | 98 ± 5 | 1063 ± 161 | 1811 ± 290 | 39.5 | 0.362 | 0.058 |
| G3 | 98 ± 5 | 852 ± 94 | 1121 ± 246 | 63.9 | 0.975 | 0.005 |
| G4 | 98 ± 4 | 762 ± 114 | 1255 ± 270 | 59.1 | 0.880 | 0.008 |
| G5 | 98 ± 6 | 665 ± 123 | 854 ± 165 | 73.3 | 0.680 | 0.001 |
| G6 | 98 ± 3 | 742 ± 161 | 669 ± 172 | 79.8 | 0.841 | 0.001 |
| G7 | 98 ± 6 | 922 ± 108 | 1373 ± 306 | 55.0 | 0.638 | 0.015 |

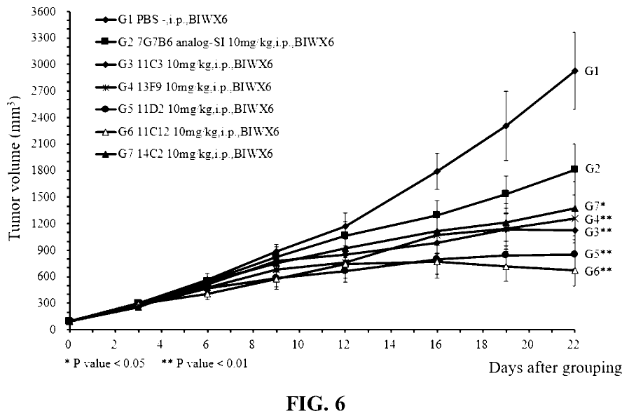

The tumor volume of mice in different groups treated with the antibodies or PBS are shown in FIG. 6. The anti-IL2RA antibodies (G3-G7) showed a better tumor inhibitory effect compared with PBS in the control groups (G1) and the positive control 7G7B6 analog-SI (G2) at a dose level of 10 mg/kg.

### Example 6. Anti-Tumor Activity in GL261 Xenograft Model

About 1×10⁶ mouse glioma GL261 cells were injected subcutaneously in each B-hIL2RA mouse. When the tumors in the mice reached a volume of about 80-90 mm³, the mice were randomly placed into different groups based on tumor size. The mice were then injected with PBS or anti-IL2RA antibodies by intraperitoneal (i.p.) administration. Details are shown in the table below.

TABLE 9
Group assignment

| | No. of | | | | | Total No. of |
|---|---|---|---|---|---|---|
| Group | mice | Antibody | Dosage | Route | Frequency | administration |
| G1 | 6 | PBS | — | i.p. | BIW | 6 |
| G2 | 6 | 7B5 | 10 mg/kg | i.p. | BIW | 6 |
| G3 | 6 | 5D9 | 10 mg/kg | i.p. | BIW | 6 |
| G4 | 6 | 7G7B6 | 10 mg/kg | i.p. | BIW | 6 |
| | | analog | | | | |

The weights of mice in different groups all increased. On the day of group assignment (Day 0), the average weight of each group was in the range of 20.5 g-20.9 g. At the end of the experiment (Day 14), the average weight of each group was in the range of 23.3 g-25.0 g. The average weight change of each group was in the range of 113.5%-119.4%. The results showed that the tested antibodies were well tolerated and were not obviously toxic to the mice.

The table below summarizes the results for this experiment, including the tumor volumes on the day of grouping (Day 0), 7 days after grouping (Day 7) and 14 days after grouping (Day 14); Tumor Growth Inhibition value (TGI); and the statistical differences (P value) of tumor volume and body weight between the treatment and control groups.

TABLE 10
Tumor size changes

| | | | | | P value | |
|---|---|---|---|---|---|---|
| | Tumor volume (mm³) | | | | Body | Tumor |
| Group | Day 0 | Day 7 | Day 14 | TGI (%) | weight | volume |
| G1 | 94 ± 3 | 1282 ± 62 | 3069 ± 289 | NA | NA | NA |
| G2 | 94 ± 4 | 754 ± 198 | 1197 ± 322 | 62.9 | 0.106 | 0.001 |
| G3 | 94 ± 3 | 837 ± 100 | 1394 ± 242 | 56.3 | 0.238 | 0.001 |
| G4 | 94 ± 3 | 837 ± 79 | 1346 ± 204 | 57.9 | 0.475 | 0.001 |

The treatment groups (G2-G4) showed better tumor inhibitory effects compared with the control groups (G1), which were treated with PBS. In addition, 7B5 showed a better tumor inhibitory effect compared with the positive control 7G7B6 analog and the tumor inhibitory effect of 5D9 was basically equivalent to that of the positive control 7G7B6 analog.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to interleukin-2 receptor alpha chain (IL2RA), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 43, 44, 45, respectively;
(2) the selected VH CDRs 1, 2, 3 amino acid sequences are set forth in SEQ ID NOs: 4, 5, 6, respectively, and the selected VL CDRs 1, 2, 3 amino acid sequences are set forth in SEQ ID NOs: 46, 47, 48, respectively;
(3) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 49, 50, 51, respectively;
(4) the selected VH CDRs 1, 2, 3 amino acid sequences are set forth in SEQ ID NOs: 10, 11, 12, respectively, and the selected VL CDRs 1, 2, 3 amino acid sequences are set forth in SEQ ID NOs: 49, 50, 51, respectively;
(5) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 49, 50, 51, respectively;
(6) the selected VH CDRs 1, 2, 3 amino acid sequences are set forth in SEQ ID NOs: 16, 17, 18, respectively, and the selected VL CDRs 1, 2, 3 amino acid sequences are set forth in SEQ ID NOs: 49, 50, 51, respectively;
(7) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 49, 50, 51, respectively;
(8) the selected VH CDRs 1, 2, 3 amino acid sequences are set forth in SEQ ID NOs: 22, 23, 24, respectively, and the selected VL CDRs 1, 2, 3 amino acid sequences are set forth in SEQ ID NOs: 43, 44, 45, respectively;
(9) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 46, 47, 48, respectively;
(10) the selected VH CDRs 1, 2, 3 amino acid sequences are set forth in SEQ ID NOs: 28, 29, 30, respectively, and the selected VL CDRs 1, 2, 3 amino acid sequences are set forth in SEQ ID NOs: 49, 50, 51, respectively;
(11) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 49, 50, 51, respectively;
(12) the selected VH CDRs 1, 2, 3 amino acid sequences are set forth in SEQ ID NOs: 34, 35, 36, respectively, and the selected VL CDRs 1, 2, 3 amino acid sequences are set forth in SEQ ID NOs: 49, 50, 51, respectively;
(13) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 49, 50, 51, respectively; and
(14) the selected VH CDRs 1, 2, 3 amino acid sequences are set forth in SEQ ID NOs: 40, 41, 42, respectively, and the selected VL CDRs 1, 2, 3 amino acid sequences are set forth in SEQ ID NOs: 49, 50, 51, respectively.

2. The antibody or antigen-binding fragment thereof of claim 1, wherein the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 43, 44, and 45, respectively according to Kabat definition.

3. The antibody or antigen-binding fragment thereof of claim 1, wherein the VH comprises CDRs 1, 2, 3 with the amino acid sequences set forth in SEQ ID NOs: 4, 5, and 6, respectively, and the VL comprises CDRs 1, 2, 3 with the amino acid sequences set forth in SEQ ID NOs: 46, 47, and 48, respectively according to Kabat definition.

4. The antibody or antigen-binding fragment thereof of claim 1, wherein the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 49, 50, and 51, respectively according to Kabat definition.

5. The antibody or antigen-binding fragment thereof of claim 1, wherein the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 49, 50, and 51, respectively according to Kabat definition.

6. The antibody or antigen-binding fragment thereof of claim 1, wherein the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 49, 50, and 51, respectively according to Kabat definition.

7. The antibody or antigen-binding fragment thereof of claim 1, wherein the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 49, 50, and 51, respectively according to Kabat definition.

8. The antibody or antigen-binding fragment thereof of claim 1, wherein the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 49, 50, and 51, respectively according to Kabat definition.

9. The antibody or antigen-binding fragment thereof of any one of claims 1-8, wherein the antibody or antigen-binding fragment specifically binds to human IL2RA or monkey IL2RA.

10. The antibody or antigen-binding fragment thereof of any one of claims 1-9, wherein the antibody or antigen-binding fragment is a human or humanized antibody or antigen-binding fragment thereof (e.g., a human IgG1 antibody).

11. The antibody or antigen-binding fragment thereof of any one of claims 1-10, wherein the antibody or antigen-binding fragment is a single-chain variable fragment (scFv) or a multi-specific antibody (e.g., a bispecific antibody).

12. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 53 binds to IL2RA;
(2) an immunoglobulin light chain or a fragment thereof comprising a VL comprising CDRs 1, 2, and 3 comprising the amino acid sequences set forth in SEQ ID NOs: 43, 44, and 45, respectively, and wherein the VL, when paired with a VH comprising the amino acid sequence set forth in SEQ ID NO: 52 binds to IL2RA;
(3) an immunoglobulin heavy chain or a fragment thereof comprising a heavy chain variable region (VH) comprising CDRs 1, 2, and 3 comprising the amino acid sequences set forth in SEQ ID NOs: 4, 5, and 6, respectively, and wherein the VH, when paired with a light chain variable region (VL) comprising the amino acid sequence set forth in SEQ ID NO: 55 binds to IL2RA;
(4) an immunoglobulin light chain or a fragment thereof comprising a VL comprising CDRs 1, 2, and 3 comprising the amino acid sequences set forth in SEQ ID NOs: 46, 47, and 48, respectively, and wherein the VL, when paired with a VH comprising the amino acid sequence set forth in SEQ ID NO: 54 binds to IL2RA;
(5) an immunoglobulin heavy chain or a fragment thereof comprising a heavy chain variable region (VH) comprising CDRs 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 61 binds to IL2RA;
(6) an immunoglobulin light chain or a fragment thereof comprising a VL comprising CDRs 1, 2, and 3 comprising the amino acid sequences set forth in SEQ ID NOs: 49, 50, and 51, respectively, and wherein the VL, when paired with a VH comprising the amino acid sequence set forth in SEQ ID NO: 56 binds to IL2RA;
(7) an immunoglobulin heavy chain or a fragment thereof comprising a heavy chain variable region (VH) comprising CDRs 1, 2, and 3 comprising the amino acid sequences set forth in SEQ ID NOs: 10, 11, and 12, respectively, and wherein the VH, when paired with a light chain variable region (VL) comprising the amino acid sequence set forth in SEQ ID NO: 61 binds to IL2RA;
(8) an immunoglobulin light chain or a fragment thereof comprising a VL comprising CDRs 1, 2, and 3 comprising the amino acid sequences set forth in SEQ ID NOs: 49, 50, and 51, respectively, and wherein the VL, when paired with a VH comprising the amino acid sequence set forth in SEQ ID NO: 57 binds to IL2RA;
(9) an immunoglobulin heavy chain or a fragment thereof comprising a heavy chain variable region (VH) comprising CDRs 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 61 binds to IL2RA;
(10) an immunoglobulin light chain or a fragment thereof comprising a VL comprising CDRs 1, 2, and 3 comprising the amino acid sequences set forth in SEQ ID NOs: 49, 50, and 51, respectively, and wherein the VL, when paired with a VH comprising the amino acid sequence set forth in SEQ ID NO: 58 binds to IL2RA;
(11) an immunoglobulin heavy chain or a fragment thereof comprising a heavy chain variable region (VH) comprising CDRs 1, 2, and 3 comprising the amino acid sequences set forth in SEQ ID NOs: 16, 17, and 18, respectively, and wherein the VH, when paired with a light chain variable region (VL) comprising the amino acid sequence set forth in SEQ ID NO: 61 binds to IL2RA;
(12) an immunoglobulin light chain or a fragment thereof comprising a VL comprising CDRs 1, 2, and 3 comprising the amino acid sequences set forth in SEQ ID NOs: 49, 50, and 51, respectively, and wherein the VL, when paired with a VH comprising the amino acid sequence set forth in SEQ ID NO: 59 binds to IL2RA;
(13) an immunoglobulin heavy chain or a fragment thereof comprising a heavy chain variable region (VH) comprising CDRs 1, 2, and 3 comprising the amino acid sequences set forth in SEQ ID NOs: 19, 20, and 21, respectively, and wherein the VH, when paired with a light chain variable region (VL) comprising the amino acid sequence set forth in SEQ ID NO: 61 binds to IL2RA;
(14) an immunoglobulin light chain or a fragment thereof comprising a VL comprising CDRs 1, 2, and 3 comprising the amino acid sequences set forth in SEQ ID NOs: 49, 50, and 51, respectively, and wherein the VL, when paired with a VH comprising the amino acid sequence set forth in SEQ ID NO: 60 binds to IL2RA;
(15) an immunoglobulin heavy chain or a fragment thereof comprising a heavy chain variable region (VH) comprising CDRs 1, 2, and 3 comprising the amino acid sequences set forth in SEQ ID NOs: 22, 23, and 24, respectively, and wherein the VH, when paired with a light chain variable region (VL) comprising the amino acid sequence set forth in SEQ ID NO: 53 binds to IL2RA;
(16) an immunoglobulin heavy chain or a fragment thereof comprising a heavy chain variable region (VH) comprising CDRs 1, 2, and 3 comprising the amino acid sequences set forth in SEQ ID NOs: 25, 26, and 27, respectively, and wherein the VH, when paired with a light chain variable region (VL) comprising the amino acid sequence set forth in SEQ ID NO: 55 binds to IL2RA;
(17) an immunoglobulin heavy chain or a fragment thereof comprising a heavy chain variable region (VH) comprising CDRs 1, 2, and 3 comprising the amino acid sequences set forth in SEQ ID NOs: 28, 29, and 30, respectively, and wherein the VH, when paired with a light chain variable region (VL) comprising the amino acid sequence set forth in SEQ ID NO: 61 binds to IL2RA;
(18) an immunoglobulin heavy chain or a fragment thereof comprising a heavy chain variable region (VH) comprising CDRs 1, 2, and 3 comprising the amino acid sequences set forth in SEQ ID NOs: 31, 32, and 33, respectively, and wherein the VH, when paired with a light chain variable region (VL) comprising the amino acid sequence set forth in SEQ ID NO: 61 binds to IL2RA;
(19) an immunoglobulin heavy chain or a fragment thereof comprising a heavy chain variable region (VH) comprising CDRs 1, 2, and 3 comprising the amino acid sequences set forth in SEQ ID NOs: 34, 35, and 36, respectively, and wherein the VH, when paired with a light chain variable region (VL) comprising the amino acid sequence set forth in SEQ ID NO: 61 binds to IL2RA;
(20) an immunoglobulin heavy chain or a fragment thereof comprising a heavy chain variable region (VH) comprising CDRs 1, 2, and 3 comprising the amino acid sequences set forth in SEQ ID NOs: 37, 38, and 39, respectively, and wherein the VH, when paired with a light chain variable region (VL) comprising the amino acid sequence set forth in SEQ ID NO: 61 binds to IL2RA; or
(21) an immunoglobulin heavy chain or a fragment thereof comprising a heavy chain variable region (VH) comprising CDRs 1, 2, and 3 comprising the amino acid sequences set forth in SEQ ID NOs: 40, 41, and 42, respectively, and wherein the VH, when paired with a light chain variable region (VL) comprising the amino acid sequence set forth in SEQ ID NO: 61 binds to IL2RA.

13. The nucleic acid of claim 12, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively, or SEQ ID NOs: 22, 23, and 24, respectively.

14. The nucleic acid of claim 12, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 43, 44, and 45, respectively.

15. The nucleic acid of claim 12, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4, 5, and 6, respectively, or SEQ ID NOs: 25, 26, and 27, respectively.

16. The nucleic acid of claim 12, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 46, 47, and 48, respectively.

17. The nucleic acid of claim 12, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively, or SEQ ID NOs: 28, 29, and 30, respectively.

18. The nucleic acid of claim 12, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10, 11, and 12, respectively, or SEQ ID NOs: 31, 32, and 33, respectively.

19. The nucleic acid of claim 12, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively, or SEQ ID NOs: 34, 35, and 36, respectively.

20. The nucleic acid of claim 12, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16, 17, and 18, respectively, or SEQ ID NOs: 37, 38, and 39, respectively.

21. The nucleic acid of claim 12, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19, 20, and 21, respectively, or SEQ ID NOs: 40, 41, and 42, respectively.

22. The nucleic acid of claim 12, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 49, 50, and 51, respectively.

23. The nucleic acid of any one of claims 12-22, wherein the VH when paired with a VL specifically binds to human IL2RA, or the VL when paired with a VH specifically binds to human IL2RA or monkey IL2RA.

24. The nucleic acid of any one of claims 12-23, wherein the immunoglobulin heavy chain or the fragment thereof is a human or humanized immunoglobulin heavy chain or a fragment thereof (e.g., a human IgG1 heavy chain or a fragment thereof), and the immunoglobulin light chain or the fragment thereof is a human or humanized immunoglobulin light chain or a fragment thereof.

25. The nucleic acid of any one of claims 12-24, wherein the nucleic acid encodes a single-chain variable fragment (scFv), a multi-specific antibody (e.g., a bispecific antibody) or a chimeric antigen receptor (CAR).

26. The nucleic acid of any one of claims 12-25, wherein the nucleic acid is cDNA.

27. A vector comprising one or more of the nucleic acids of any one of claims 12-26.

28. A vector comprising two of the nucleic acids of any one of claims 12-26, wherein the vector encodes the VH region and the VL region that together bind to IL2RA.

29. A pair of vectors, wherein each vector comprises one of the nucleic acids of any one of claims 12-26, wherein together the pair of vectors encodes the VH region and the VL region that together bind to IL2RA.

30. A cell comprising the vector of claim 27 or 28, or the pair of vectors of claim 29.

31. The cell of claim 30, wherein the cell is a CHO cell.

32. A cell comprising one or more of the nucleic acids of any one of claims 12-26.

33. A cell comprising two of the nucleic acids of any one of claims 12-26.

34. The cell of claim 33, wherein the two nucleic acids together encode the VH region and the VL region that together bind to IL2RA.

35. A method of producing an antibody or an antigen-binding fragment thereof, the method comprising
(a) culturing the cell of any one of claims 30-34 under conditions sufficient for the cell to produce the antibody or the antigen-binding fragment; and
(b) collecting the antibody or the antigen-binding fragment produced by the cell.

36. An antibody or antigen-binding fragment thereof that binds to IL2RA comprising a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 52, and the selected VL sequence is SEQ ID NO: 53;
(2) the selected VH sequence is SEQ ID NO: 54, and the selected VL sequence is SEQ ID NO: 55;
(3) the selected VH sequence is SEQ ID NO: 56, and the selected VL sequence is SEQ ID NO: 61;
(4) the selected VH sequence is SEQ ID NO: 57, and the selected VL sequence is SEQ ID NO: 61;
(5) the selected VH sequence is SEQ ID NO: 58, and the selected VL sequence is SEQ ID NO: 61;
(6) the selected VH sequence is SEQ ID NO: 59, and the selected VL sequence is SEQ ID NO: 61; and
(7) the selected VH sequence is SEQ ID NO: 60, and the selected VL sequence is SEQ ID NO: 61.

37. The antibody or antigen-binding fragment thereof of claim 36, wherein the VH comprises the sequence of SEQ ID NO: 52 and the VL comprises the sequence of SEQ ID NO: 53.

38. The antibody or antigen-binding fragment thereof of claim 36, wherein the VH comprises the sequence of SEQ ID NO: 54 and the VL comprises the sequence of SEQ ID NO: 55.

39. The antibody or antigen-binding fragment thereof of claim 36, wherein the VH comprises the sequence of SEQ ID NO: 56 and the VL comprises the sequence of SEQ ID NO: 61.

40. The antibody or antigen-binding fragment thereof of claim 36, wherein the VH comprises the sequence of SEQ ID NO: 57 and the VL comprises the sequence of SEQ ID NO: 61.

41. The antibody or antigen-binding fragment thereof of claim 36, wherein the VH comprises the sequence of SEQ ID NO: 58 and the VL comprises the sequence of SEQ ID NO: 61.

42. The antibody or antigen-binding fragment thereof of claim 36, wherein the VH comprises the sequence of SEQ ID NO: 59 and the VL comprises the sequence of SEQ ID NO: 61.

43. The antibody or antigen-binding fragment thereof of claim 36, wherein the VH comprises the sequence of SEQ ID NO: 60 and the VL comprises the sequence of SEQ ID NO: 61.

44. The antibody or antigen-binding fragment thereof of any one of claims 36-43, wherein the antibody or antigen-binding fragment specifically binds to human IL2RA or monkey IL2RA.

45. The antibody or antigen-binding fragment thereof of any one of claims 36-44, wherein the antibody or antigen-binding fragment is a human or humanized antibody or antigen-binding fragment thereof.

46. The antibody or antigen-binding fragment thereof of any one of claims 36-45, wherein the antibody or antigen-binding fragment is a single-chain variable fragment (scFv) or a multi-specific antibody (e.g., a bispecific antibody).

47. An antibody or antigen-binding fragment thereof that cross-competes with the antibody or antigen-binding fragment thereof of any one of claims 1-11 and 36-46.

48. An antibody or antigen-binding fragment thereof that binds to IL2RA comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 52, and the selected VL sequence is SEQ ID NO: 53;
(2) the selected VH sequence is SEQ ID NO: 54, and the selected VL sequence is SEQ ID NO: 55;
(3) the selected VH sequence is SEQ ID NO: 56, and the selected VL sequence is SEQ ID NO: 61;
(4) the selected VH sequence is SEQ ID NO: 57, and the selected VL sequence is SEQ ID NO: 61;
(5) the selected VH sequence is SEQ ID NO: 58, and the selected VL sequence is SEQ ID NO: 61;
(6) the selected VH sequence is SEQ ID NO: 59, and the selected VL sequence is SEQ ID NO: 61; and
(7) the selected VH sequence is SEQ ID NO: 60, and the selected VL sequence is SEQ ID NO: 61.

49. An antibody-drug conjugate comprising the antibody or antigen-binding fragment thereof of any one of claims 1-11 and 36-48 covalently bound to a therapeutic agent.

50. The antibody drug conjugate of claim 49, wherein the therapeutic agent is a cytotoxic or cytostatic agent.

51. A method of treating a subject having cancer, the method comprising administering a therapeutically effective amount of a composition comprising the antibody or antigen-binding fragment thereof of any one of claims 1-11 and 36-48, or the antibody-drug conjugate of claim 49 or 50, to the subject.

52. The method of claim 51, wherein the subject has a solid tumor, brain cancer, lung cancer, melanoma, prostate cancer, esophageal squamous cell carcinoma, leukemia, lymphoma, ovarian cancer, colorectal cancer, head and neck cancer, bladder cancer, renal cancer and breast cancer.

53. The method of claim 51 or 52, further comprising administering a therapeutically effective amount of an anti-OX40 antibody, an anti-PD1 antibody, an anti-PDL1 antibody, an anti-PDL2 antibody, an anti-LAG-3 antibody, an anti-TIGIT antibody, an anti-CTLA-4 antibody, an anti-GITR antibody, an anti-TIM-3 antibody, an anti-4-1BB antibody, and/or an anti-CD40 antibody, to the subject.

54. A method of decreasing the rate of tumor growth, the method comprising contacting a tumor cell with an effective amount of a composition comprising an antibody or antigen-binding fragment thereof of any one of claims 1-11 and 36-48, or the antibody-drug conjugate of claim 49 or 50.

55. A method of killing a tumor cell, the method comprising contacting a tumor cell with an effective amount of a composition comprising the antibody or antigen-binding fragment thereof of any one of claims 1-11 and 36-48, or the antibody-drug conjugate of claim 49 or 50.

56. A pharmaceutical composition comprising the antibody or antigen-binding fragment thereof of any one of claims 1-11 and 36-48, and a pharmaceutically acceptable carrier.

57. A pharmaceutical composition comprising the antibody drug conjugate of claim 49 or 50, and a pharmaceutically acceptable carrier.

58. A chimeric antigen receptor (CAR) comprising the antibody or antigen-binding fragment thereof of any one of claims 1-11 and 36-48.
